(19) United States
(12) Patent Application Publication
(10) Pub. No.: US 2024/0058475 A1
(43) Pub. Date: Feb. 22, 2024

(54) METHODS AND COMPOSITIONS FOR TREATING MUSCULAR DYSTROPHY

(71) Applicant: Sardocor Corp., Lexington, MA (US)

(72) Inventors: Roger J. Hajjar, Lexington, MA (US); Ronald A. Li, Hong Kong (HK); On Tik Wong, Hong Kong (HK)

(21) Appl. No.: 18/260,456

(22) PCT Filed: Jan. 6, 2022
(86) PCT No.: PCT/US2022/011429

§ 371 Date: Jul. 5, 2023

Related U.S. Application Data
(60) Provisional application No. 63/135,121, filed on Jan. 8, 2021.

(51) Int. Cl. A61K 48/00; A61P 21/00

(52) U.S. Cl. CPC A61K 48/005; A61P 21/00

(57) ABSTRACT

Some embodiments of the methods and compositions provided herein relate to treating, inhibiting or ameliorating a skeletal muscular dystrophy with a polynucleotide encoding a SERCA2a polypeptide. In some embodiments, the muscular dystrophy comprises Duchenne muscular dystrophy (DMD) or Becker's muscular dystrophy (BMD). In some embodiments, the polynucleotide comprises a viral vector, such as an adeno-associated viral (AAV) vector. More embodiments include methods and compositions to screen for a therapeutic agent to treat, inhibit or ameliorate a skeletal muscular dystrophy in which the screen comprises an in vitro ventricular cardiac tissue model.

### RELATES APPLICATIONS

This application claims priority to U.S. Prov. App. No. 63/135,121 filed Jan. 8, 2021 entitles “METHODS AND COMPOSITIONS FOR TREATING MUSCULAR DYSTROPHY” which is incorporated by reference herein in its entirety.

### STATEMENT REGARDING FEDERALLY SPONSORED R&D

This invention was made with government support under AR-70517 and AR-69107 awarded by National Institutes of Health. The government has certain rights in the invention.

### FIELD OF THE INVENTION

Some embodiments of the methods and compositions provided herein relate to treating, inhibiting (preventing) or ameliorating a skeletal muscular dystrophy with a polynucleotide encoding a SERCA2a polypeptide. In some embodiments, the muscular dystrophy comprises Duchenne muscular dystrophy (DMD) or Becker's muscular dystrophy (BMD). In some embodiments, the polynucleotide comprises a viral vector, such as an adeno-associated viral (AAV) vector. More embodiments include methods and compositions to screen for a therapeutic agent to treat, inhibit (prevent) or ameliorate a skeletal muscular dystrophy in which the screen comprises an in vitro ventricular cardiac tissue model.

### BACKGROUND OF THE INVENTION

The loss of sub-sarcolemmal cytoskeletal protein dystrophin leads to Duchenne muscular dystrophy (DMD), a chronic disease characterized by degeneration, necrosis, and fatty fibrosis of the heart and skeletal muscle. Numerous gene replacement and gene repair studies have been performed to restore dystrophin expression. While preclinical data are compelling, the potential immunogenicity of newly expressed dystrophin remains a concern. The complexity of thousands of disease-causing mutations in the dystrophin gene creates additional challenges to dystrophin repair gene therapy. Dystrophin-independent disease-modifying gene therapy offers an opportunity to treat all DMD patients without the complication of dystrophin immunity.

### SUMMARY OF THE INVENTION

Some embodiments of the methods and compositions provided herein include a method of treating, inhibiting or ameliorating a skeletal muscular dystrophy in a subject, comprising: administering a polynucleotide comprising a nucleic acid encoding a sarcoplasmic/endoplasmic reticulum calcium ATPase (SERCA) polypeptide to the subject.

In some embodiments, the muscular dystrophy comprises a systemic dystrophin deficiency in the subject. In some embodiments, the muscular dystrophy is selected from Duchenne muscular dystrophy (DMD) and Becker's muscular dystrophy (BMD). In some embodiments, the muscular dystrophy comprises DMD.

In some embodiments, the skeletal muscular dystrophy comprises myocardial remodeling and/or fibrosis.

In some embodiments, the treating, inhibiting or ameliorating reduces myocardial remodeling and/or fibrosis in the subject compared to a subject not administered the polynucleotide.

In some embodiments, the SERCA polypeptide comprises a SERCA2a polypeptide.

In some embodiments, the polynucleotide comprises a vector. In some embodiments, the vector is selected from an adeno-associated virus (AAV) vector, a lentivirus vector, and a retrovirus vector In some embodiments, the vector comprises an AAV vector. In some embodiments, the AAV vector encodes an AAV or fragment thereof having a serotype selected from any one of AAV serotypes 1-12. In some embodiments, the AAV vector encodes an AAV or fragment thereof having a serotype selected from an AAV serotype-1 (AAV1), and an AAV serotype-9 (AAV9).

In some embodiments, the polynucleotide comprises a promoter operably linked to the nucleic acid encoding the SERCA polypeptide. In some embodiments, the promoter comprises a constitutive promoter. In some embodiments, the promoter comprises a cytomegalovirus (CMV) promoter.

In some embodiments, a viral capsid comprises the polynucleotide. In some embodiments, the polynucleotide comprises a nucleic acid encoding the viral capsid.

Some embodiments also include determining the presence or absence in the subject of an antibody against an AAV serotype.

In some embodiments, the administering a polynucleotide comprises systemic administration. In some embodiments, the administering a polynucleotide comprises intravenous administration. In some embodiments, the administering a polynucleotide comprises intracoronary infusion. In some embodiments, the administering a polynucleotide comprises in utero administration.

In some embodiments, the administering a polynucleotide consists of a single dose of the polynucleotide.

In some embodiments, the polynucleotide comprises a viral vector, and wherein the administration comprises a dose of the polynucleotide within a range from about 1×10⁸ viral genome particles to about 1×10¹⁵ viral genome particles. In some embodiments, the dose is within a range from about 1×10¹³ viral genome particles to about 9×10¹³ viral genome particles.

Some embodiments also include administering a vasodilator. In some embodiments, the vasodilator is administered prior to administering a polynucleotide. In some embodiments, the vasodilator is administered concurrently with administering a polynucleotide. In some embodiments, the vasodilator comprises nitroglycerin.

In some embodiments, the polynucleotide is administered prior to an onset of muscle tissue damage. In some embodiments, the muscle tissue damage is predicted to results from the skeletal muscular dystrophy. In some embodiments, the muscle tissue damage can be measured by muscle histology.

In some embodiments, the subject is in utero.

In some embodiments, the subject is neonate.

In some embodiments, the subject is at least 3 years of age. In some embodiments, the subject is at least 5 years of age. In some embodiments, the subject is at least 10 years of age.

In some embodiments, the subject is not more than 20, or not more than 15, years of age.

In some embodiments, the subject is at least 10 years of age, and not more than 20 years of age.

In some embodiments, the subject is non-ambulatory.

In some embodiments, the subject has decreased cardiac function compared to the cardiac function of a subject not having a muscular dystrophy.

In some embodiments, the subject is mammalian. In some embodiments, the subject is human. In some embodiments, the subject is male.

In some embodiments, the treatment provides an improvement in a symptom or measure of the skeletal muscular dystrophy in the subject compared to the symptom or measure in an untreated subject for a period following administration of the polynucleotide of at least 1, 2, 3, 6, 9, or 12 months. In some embodiments, the period is at least 1, 2, 3, 4, 5, 6, 7, 8, 9 or 10 years.

In some embodiments, the treatment provides an improvement in cardiac tissue or cardiac function in the subject compared to an untreated subject.

In some embodiments, the treatment provides an improvement in skeletal muscle tissue or skeletal muscle function in the subject compared to an untreated subject.

In some embodiments, the treatment provides an improvement in ventricular function in the subject compared to ventricular function in an untreated subject. In some embodiments, the improvement in ventricular function comprises an improvement in a parameter selected from the group consisting of a change from baseline in left ventricular structure and function as assessed by late gadolinium enhancement (LGE) cardiac MRI including left ventricular ejection fraction, end-diastolic volume, end-systolic volume, stroke volume and/or circumferential strain; regional wall thickness; left ventricular LGE expressed as a percent of left ventricular mass; left ventricular viable mass; number of left ventricular segments with LGE; and composite outcome in change from baseline in LV function (LVESV).

In some embodiments, the improvement comprises an improvement in a parameter selected from the group consisting of a change from baseline in left ventricular structure and function as assessed by late gadolinium enhancement (LGE) cardiac MRI including left ventricular ejection fraction, end-diastolic volume, end-systolic volume, stroke volume and/or circumferential strain; regional wall thickness; left ventricular LGE expressed as a percent of left ventricular mass; left ventricular viable mass; number of left ventricular segments with LGE; composite outcome in change from baseline in LV function (LVESV), PUL 2.0, pulmonary function selected from quality of life and a terminal event; and a change from baseline in any one of: (a) skeletal muscle function as assessed by PUL 2.0, grip strength, key and tip-to-tip pinch strength, elbow flexion strength and, if ambulatory, 10-Meter Walk/Run Time (10MWRT), incidence of loss of ambulation defined as 10MWRT>30 seconds and North Star Ambulatory Assessment (NSAA); (b) pulmonary function as assessed by slow vital capacity (SVC), forced expiratory volume in one second (FEV1), forced vital capacity (FVC), peak expiratory flow (PEF), maximum inspiratory pressure (MIP), maximum expiratory pressure (MEP), peak cough flow (PCF) and inspiratory flow reserve (IFR); or (c) quality of life as assessed by DMD UL-PROM and PODCI.

Some embodiments also include measuring the improvement in the subject after the period.

Some embodiments of the methods and compositions provided herein include use of a polynucleotide comprising a nucleic acid encoding a sarcoplasmic/endoplasmic reticulum calcium ATPase (SERCA) polypeptide to treat, inhibit or ameliorate a skeletal muscular dystrophy in a subject.

Some embodiments of the methods and compositions provided herein include use of a polynucleotide comprising a nucleic acid encoding a sarcoplasmic/endoplasmic reticulum calcium ATPase (SERCA) polypeptide in the manufacture of a medicament to treat, inhibit or ameliorate a skeletal muscular dystrophy in a subject.

In some embodiments, the muscular dystrophy comprises a systemic dystrophin deficiency in the subject. In some embodiments, the muscular dystrophy is selected from Duchenne muscular dystrophy (DMD) and Becker's muscular dystrophy (BMD). In some embodiments, the muscular dystrophy comprises DMD. In some embodiments, the skeletal muscular dystrophy comprises myocardial remodeling and/or fibrosis.

In some embodiments, the SERCA polypeptide comprises a SERCA2a polypeptide.

In some embodiments, the polynucleotide comprises a vector. In some embodiments, the vector is selected from an adeno-associated virus (AAV) vector, a lentivirus vector, and a retrovirus vector. In some embodiments, the vector comprises an AAV vector. In some embodiments, the AAV vector encodes an AAV or fragment thereof having a serotype selected from any one of AAV serotypes 1-12. In some embodiments, the AAV vector encodes an AAV or fragment thereof having a serotype selected from an AAV serotype-1 (AAV1), and an AAV serotype-9 (AAV9).

In some embodiments, the polynucleotide comprises a promoter operably linked to the nucleic acid encoding the SERCA polypeptide. In some embodiments, the promoter comprises a constitutive promoter. In some embodiments, the promoter comprises a cytomegalovirus (CMV) promoter.

In some embodiments, a viral capsid comprises the polynucleotide. In some embodiments, the polynucleotide comprises a nucleic acid encoding the viral capsid.

In some embodiments, the polynucleotide is adapted for systemic administration. In some embodiments, the polynucleotide is adapted for intravenous administration. In some embodiments, the polynucleotide is adapted for administration by intracoronary infusion. In some embodiments, the polynucleotide is adapted for in utero administration.

Some embodiments include any one of the foregoing uses in combination with a vasodilator. In some embodiments, the vasodilator comprises nitroglycerin.

In some embodiments, the subject is mammalian. In some embodiments, the subject is human.

In some embodiments, the subject is in utero.

In some embodiments, the subject is an infant.

In some embodiments, the subject is neonate.

In some embodiments, the subject is at least 3 years of age. In some embodiments, the subject is at least 5 years of age. In some embodiments, the subject is at least 10 years of age.

In some embodiments, the subject is not more than 20, or not more than 15, years of age.

In some embodiments, the subject is male.

Some embodiments of the methods and compositions provided herein include a method for screening for a therapeutic agent to treat, inhibit or ameliorate a skeletal muscular dystrophy in a patient, comprising: (a) contacting a test agent with a ventricular cardiac tissue strip; (b) measuring a contractile amplitude of the ventricular cardiac tissue strip contacted with the test agent; (c) comparing the contractile amplitude of the ventricular cardiac tissue strip contacted with the test agent with a contractile amplitude of a ventricular cardiac tissue strip not contacted with the test agent; and (d) determining that the test agent comprises a therapeutic agent based on the comparison.

In some embodiments, the ventricular cardiac tissue strip is obtained by: (i) differentiating a population of induced pluripotent stem cells to obtain a plurality of cardiospheres comprising a plurality of the ventricular cardiomyocytes; (ii) dissociating the plurality of cardiospheres to obtain a plurality of cardiac cells; and (iii) contacting the plurality of cardiac cells with a population of fibroblasts in the presence of collagen under conditions to obtain the ventricular cardiac tissue strip.

Some embodiments also include obtaining the ventricular cardiac tissue strip, comprising: (i) differentiating a population of induced pluripotent stem cells to obtain a plurality of cardiospheres comprising a plurality of the ventricular cardiomyocytes; (ii) dissociating the plurality of cardiospheres to obtain a plurality of cardiac cells; and (iii) contacting the plurality of cardiac cells with a population of fibroblasts in the presence of collagen under conditions to obtain the ventricular cardiac tissue strip.

In some embodiments, the population of induced pluripotent stem cells is obtained from a subject having Duchenne muscular dystrophy (DMD).

In some embodiments, (a) is performed for a period of at least 1 hour. In some embodiments, (a) is performed for a period of at least 1 day.

In some embodiments, the ventricular cardiac tissue strip is stimulated by an electrical field having a frequency. In some embodiments, (b) is performed at more than one frequency.

In some embodiments, (b) comprises measuring a parameter selected from developed force, normalized developed force, rate variability, force variability, force-frequency relationship, and beta adrenergic response.

In some embodiments, the muscular dystrophy comprises a systemic dystrophin deficiency in the patient. In some embodiments, the muscular dystrophy is selected from Duchenne muscular dystrophy (DMD) and Becker's muscular dystrophy (BMD). In some embodiments, the muscular dystrophy comprises DMD.

In some embodiments, the test agent comprises a polynucleotide. In some embodiments, the polynucleotide encodes a SERCA polypeptide. In some embodiments, the SERCA polypeptide comprises a SERCA2a polypeptide.

In some embodiments, the polynucleotide comprises a vector. In some embodiments, the vector is selected from an adeno-associated virus (AAV) vector, a lentivirus vector, and a retrovirus vector. In some embodiments, the vector comprises an AAV vector. In some embodiments, the AAV vector encodes an AAV or fragment thereof having a serotype selected from any one of AAV serotypes 1-12. In some embodiments, the AAV vector encodes an AAV or fragment thereof having a serotype selected from an AAV serotype-1 (AAV1), and an AAV serotype-9 (AAV9).

In some embodiments, the polynucleotide comprises a promoter operably linked to the nucleic acid encoding the SERCA polypeptide. In some embodiments, the promoter comprises a constitutive promoter. In some embodiments, the promoter comprises a cytomegalovirus (CMV) promoter. In some embodiments, the promoter comprises an inducible promoter.

In some embodiments, the polynucleotide is packaged in a viral capsid. In some embodiments, the polynucleotide comprises a nucleic acid encoding the viral capsid.

### BRIEF DESCRIPTION OF THE DRAWINGS

FIG. 1A depicts a schematic map of a human SERCA2a encoded by an AAV vector with the CMV (cytomegalovirus) promoter; i, intron.

FIG. 1B depicts an experimental plan in which 6×10¹² vg particles/mouse of the AAV9 (serotype 9 of the AAV) vectors encoding for SERCA2a were injected into 3-month-old mice via the tail vein. Grip strength and treadmill performance were evaluated at 11 months of age. When mice reached 21 months of age, the serum CK level, grip strength, treadmill running distance, ECG, and left ventricle hemodynamics were evaluated.

FIG. 1C depicts laminin and flag immunostaining photomicrographs from the heart of wild-type, mdx, and AAV9.SERCA2a-injected mdx mice. Laminin staining reveals the basement membrane. Flag staining reveals human SERCA2a expression.

FIG. 1D depicts a whole heart lysate western blot (left panel) and a densitometry quantification (right panel) from wild-type, mdx, and AAV9.SERCA2a-injected mdx mice. Flag signal reveals human SERCA2a expression. Vinculin was the loading control. *p<0.05.

FIG. 1E depicts a sarcoplasmic/endoplasmic reticulum (SR) preparation western blot from wild-type, mdx, and AAV9.SERCA2a-injected mdx mice.

FIG. 1F depicts cardiac SR calcium uptake tracing. *p<0.05 compared to that of wild-type mice and AAV9.SERCA2a-treated mice (left panel); and the maximum rate of calcium uptake (Vₘₐₓ). *p<0.05 (right panel).

FIG. 1G depicts western blot images of SERCA2a, PLN and CSQ. Glyceraldehyde 3-phosphate dehydrogenase (GAPDH) is the loading control.

FIG. 1H depicts western blot images of nNOS, and graphs for quantification of the same.

FIG. 2A depicts an immunoblot (western) blot for detecting SERCA2a from four independent skeletal muscles in untreated and AAV9.SERCA2a-injected mdx mice. The flag tag antibody reveals human SERCA2a. Vinculin is the loading control.

FIG. 2B depicts laminin and flag immunostaining photomicrographs from four independent skeletal muscles of AAV9.SERCA2a injected mdx mice. Laminin staining reveals the basement membrane. Flag signal reveals human SERCA2a expression.

FIG. 2C depicts skeletal muscle SR calcium uptake curve, *p<0.05 compared to that of wild-type mice and AAV9.SERCA2a treated mice, ^{$}p<0.05 between wild-type mice and untreated mdx mice, and ^{#}p<0.05 between wild-type mice and all mdx mice irrespective of AAV injection (left panel); and the maximum rate of calcium uptake (Vₘₐₓ), *p<0.05 (right panel).

FIG. 3A depicts hematoxylin and eosin (H&E) and Masson trichrome staining photomicrographs of the heart from wild-type, mdx, and AAV9.SERCA2a-injected mdx mice, fibrotic tissues are stained in blue in Masson trichrome staining (left panel); and quantification of the fibrotic area in the heart (right panel).

FIG. 3B depicts an ECG evaluation of the heart rate, PR interval, QRS duration, QTc interval, Q amplitude, and cardiomyopathy index.

FIG. 3C depicts a cardiac catheter evaluation of the left ventricular end-systolic volume, dP/dt max, maximum pressure, enddiastolic volume, dP/dt min, and ejection fraction.

FIG. 3D depicts pressure-volume loops from wild-type, mdx, and AAV9.SERCA2a injected mdx mice. *p<0.05.

FIG. 3E depicts whole heart cross-section Masson trichrome staining photomicrographs from wild-type, mdx and AAV9.SERCA2a treated mdx mice at 21 months of age.

FIG. 4A depicts a forelimb grip force quantification.

FIG. 4B depicts absolute treadmill running distance (left panel) and body weight normalized running distance (right panel). *p<0.05. Data are presented as mean±standard error of the mean.

FIG. 5A depicts forelimb grip force in wild-type, mdx, and AAV9.SERCA2a treated mdx mice at 21 months of age.

FIG. 5B depicts treadmill running distance in wild-type, mdx, and AAV9.SERCA2a treated mdx mice at 21 months of age. *p<0.05. Data are presented as mean±standard error of the mean.

FIG. 5C depicts serum creatine kinase (CK) quantification from wild-type, mdx and AAV9.SERCA2a injected mdx mice at 21 months of age.

FIG. 6A depicts full-view cross-section Masson trichrome staining photomicrographs from wild-type, mdx and AAV9.SERCA2a treated mdx mice at 21 months of age.

FIG. 6B depicts high-power hematoxylin-eosin (HE) and Masson trichrome staining photomicrographs from wild-type, mdx and AAV9.SERCA2a injected mdx mice at 21 months of age.

FIG. 6C depicts quantification of fibrotic area. Sample size refers to the number of animals examined.

FIG. 6D depicts quantification of myofibers with centrally located nuclei. Sample size refers to the number of animals examined.

FIG. 6E depicts myofiber size distribution quantification. Sample size refers to the number of myofibers quantified.

FIG. 7A depicts full-view cross-section Masson trichrome staining photomicrographs from wild-type, mdx and AAV9.SERCA2a treated mdx mice at 21 months of age.

FIG. 7B depicts high-power hematoxylin-eosin (HE) and Masson trichrome staining photomicrographs from wild-type, mdx and AAV9.SERCA2a injected mdx mice at 21 months of age.

FIG. 7C depicts quantification of fibrotic area. Sample size refers to the number of animals examined.

FIG. 7D depicts quantification of myofibers with centrally located nuclei. Sample size refers to the number of animals examined.

FIG. 7E depicts myofiber size distribution quantification. Sample size refers to the number of myofibers quantified.

FIG. 8A depicts immunostaining photomicrographs (top panels) and quantification (bottom panels) for the tibialis anterior muscles from wild-type, mdx and AAV.SERCA2a treated mdx mice. Asterisk, p<0.05.

FIG. 8B depicts immunostaining photomicrographs (top panels) and quantification (bottom panels) for the quadriceps muscles from wild-type, mdx and AAV.SERCA2a treated mdx mice. Asterisk, p<0.05.

FIG. 8C depicts immunostaining photomicrographs (top panels) and quantification (bottom panels) for the gastrocnemius muscles from wild-type, mdx and AAV.SERCA2a treated mdx mice. Asterisk, p<0.05.

FIG. 9 depicts a schematic of an embodiment of a Phase 2 trial design. Abbreviations: Pre-Scr, prescreening; Scr, screening; Rand, randomization; vg, viral genomes; D, Day; W, Week; M, month.

FIG. 10 depicts an angiogram with arrows pointing to a left anterior descending artery (LAD) and a left circumflex artery (LCx) in a porcine subject.

### DETAILED DESCRIPTION

Some embodiments of the methods and compositions provided herein relate to treating, inhibiting (preventing) or ameliorating skeletal and cardiac muscular dystrophy with a polynucleotide encoding a SERCA2a polypeptide. In some embodiments, the muscular dystrophy comprises Duchenne muscular dystrophy (DMD) or Becker's muscular dystrophy (BMD). In some embodiments, the polynucleotide comprises a viral vector, such as an adeno-associated viral (AAV) vector. More embodiments include methods and compositions to screen for a therapeutic agent to treat, inhibit or ameliorate a skeletal muscular dystrophy in which the screen comprises an in vitro ventricular cardiac tissue model.

Loss of dystrophin leads to DMD. A pathogenic feature of DMD is the significant elevation of cytosolic calcium. Supra-physiological intracellular calcium triggers protein degradation, membrane damage, and eventually muscle death and dysfunction. Sarcoplasmic/endoplasmic reticulum calcium ATPase (SERCA) is a calcium pump that transports cytosolic calcium to the SR during excitation contraction coupling.

Some embodiments of the methods and compositions provided herein include systemic delivery of SERCA2a with AAV to improve calcium recycling and provide long-lasting benefits in DMD. In some embodiments, the delivery is a single dose. As disclosed herein, an AAV9 human SERCA2a vector (6×10¹² viral genome particles/mouse) was injected intravenously to 3-month-old mdx mice, a DMD model. Immunostaining and western blot showed robust human SERCA2a expression in the heart and skeletal muscle for 18 months. Concomitantly, Sarcoplasmic/endoplasmic reticulum calcium uptake was significantly improved in these tissues. SERCA2a therapy significantly enhanced grip force and treadmill performance, completely prevented myocardial fibrosis, and normalized electrocardiograms (ECGs). Cardiac catheterization showed normalization of multiple systolic and diastolic hemodynamic parameters in treated mice. Importantly, chamber dilation was completely prevented, and ejection fraction was restored to the wild-type level. These results demonstrated that a single systemic AAV9 SERCA2a therapy provided long-lasting benefits in an art-recognized animal model of DMD.

Mounting evidence suggests that cytosolic calcium overload plays a pivotal role in DMD pathogenesis.¹⁻⁴ Specifically, high levels of intracellular calcium activate calcium-sensitive calpain protease and phospholipase A2. These enzymes cause proteolysis and membrane damage. Calcium dysregulation also induces free radical production and impairs mitochondrial function. Eventually, elevated calcium leads to myofiber death and muscle dysfunction. Therefore, without being bound by any theory, the restoration of calcium homeostasis is believed to mitigate muscle disease in DMD as demonstrated by the disclosure herein.

SERCA is the calcium pump that transfers calcium from the cytosol to the SR lumen against the concentration gradient.^{5,6} In cardiac cells, SERCA accounts for more than 70% of calcium removal from the cytosol in muscle cells. Among various SERCA isoforms, SERCA1a and SERCA2a are the only members naturally expressed in adult muscle.⁵ SERCA1a is selectively expressed in skeletal muscle whereas SERC2a is expressed in both skeletal and cardiac muscle. Some embodiments provided herein include increasing SERCA2a expression thereby correcting calcium overload and attenuating both skeletal muscle disease and cardiomyopathy in DMD.

Adeno-associated virus serotype-9 (AAV9) is a vector for body-wide skeletal muscle and heart gene delivery.⁷ Systemic AAV9 therapy has yielded success in treating neuromuscular diseases in human patients.⁸ Two clinical trials have also been initiated to test systemic AAV9 gene therapy in DMD patients.^{9,10} Some embodiments disclosed herein include a single intravenous injection of a human SERCA2a AAV9 vector for lifelong disease rescue in the mdx model of DMD. Mice were treated at 3 months of age and followed until the end of their life expectancy.^{11,12} AAV9 injection resulted in body-wide muscle expression of human SERCA2a and significant enhancement of SR calcium uptake. Importantly, treatment significantly improved whole-body muscle performance and ameliorated fatal dilated cardiomyopathy. Some embodiments disclosed herein include development of dystrophin-independent gene therapy for DMD by the administration of a SERCA2a AAV vector (e.g., AAV9).

Some embodiments of the methods and compositions provided herein include aspects disclosed in U.S. Pat. App. Pub. No. 2008/0076730, U.S. Pat. No. 8,221,738, and U.S. Pat. App. Pub. No. 2017/0296790 which are each incorporated by reference in its entirety. Some embodiments of the methods and compositions provided herein include aspects disclosed in Wasala N. B. et al., (2019) Molecular Therapy 28:845-854 which is incorporated by reference in its entirety.

### Definitions

As used herein, “polynucleotide”, ““nucleic acid” or “nucleic acid molecule” have their plain and ordinary meaning in view of the whole specification and may to refer to, for example, polymers comprising deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded. In some embodiments, a nucleic acid sequence encoding a fusion protein is provided. In some alternatives, the nucleic acid is RNA or DNA.

As used herein, “coding for” or “encoding” have their plain and ordinary meaning in view of the whole specification and can refer to the property of specific sequences of nucleotides in a polynucleotide, such as a gene, a cDNA, or an mRNA, to serve as templates for synthesis of other macromolecules such as a defined sequence of amino acids. Thus, a gene codes for a protein if transcription and translation of mRNA corresponding to that gene produces the protein in a cell or other biological system.

As used herein, “vector,” “expression vector” or “construct” have their plain and ordinary meaning in view of the whole specification and refer to a nucleic acid used to introduce heterologous nucleic acids into a cell that has regulatory elements to provide expression of the heterologous nucleic acids in the cell. Vectors include but are not limited to plasmid, minicircles, yeast, and viral genomes. In some embodiments, the vector is an AAV vector, a foamy viral vector, a adenoviral vector, retroviral vector, or lentiviral vector. In some embodiments, the vector is for protein expression in a mammalian system, such as a human.

As used herein, a “promoter” has its plain and ordinary meaning in view of the whole specification and refers to a region of DNA that initiates transcription of a specific gene. The promoters can be located near the transcription start site of a gene, on the same strand and upstream on the DNA (the 5′ region of the sense strand). The promoter can be a conditional, inducible or a constitutive promoter. The promoter can be specific for bacterial, mammalian or insect cell protein expression.

As used herein, “conditional” or “inducible” has its plain and ordinary meaning in view of the whole specification and refers to a nucleic acid construct that includes a promoter that provides for gene expression in the presence of an inducer and does not substantially provide for gene expression in the absence of the inducer. Without being limiting, examples of inducible promoters for mammalian expression constructs include tetracycline, ecdysone, streptogramins, macrolides or doxycycline inducible promoters.

As used herein, “constitutive” has its plain and ordinary meaning in view of the whole specification and refers to the nucleic acid construct that includes a promoter that is constitutive, and thus provides for expression of a polypeptide that is continuously produced.

As used herein, “infusion,” “infused,” and “infusing” have their plain and ordinary meanings in view of the whole specification and refer to administration for a time period (typically a minute or more) that is substantially longer than the art recognized term of “injection” or “bolus injection,” (typically less than a minute). The flow rate of the infusion will depend at least in part on the volume administered, however the flow rate of an “infusion” is slower than that of an “injection” for the same volume.

As used herein “in conjunction with,” “in combination with,” “concurrent,” or “concurrently,” have their plain and ordinary meanings in view of the whole specification and include administration of one treatment modality in addition to another treatment modality. For example, infusion of a polynucleotide of the present invention to a subject in addition to administration of art recognized pharmaceutical composition to the same individual. As used herein, these terms include simultaneous administration, as well as administration of the treatment modalities sequentially.

As used herein, “treat” or “treatment” of disease have their plain and ordinary meanings in view of the whole specification and includes the stabilization, cure, or less than complete cure of a disease, including the halting or slowing of the progression of a disease or a sign or symptom of the disease. The term “prevention” has its plain and ordinary meaning in view of the whole specification and includes complete or incomplete prevention, or a delay of the onset of, a disease or a sign or symptom of a disease, or change the course of the disease. The terms “therapeutic,” “therapeutic effect” or “clinical effect” includes both treatment and prevention.

As used herein, the term “adeno-associated virus” or “AAV” encompasses all subtypes, serotypes and pseudotypes, as well as naturally occurring and recombinant forms. A variety of AAV serotypes and strains are known in the art and are publicly available from sources, such as the ATCC, and academic or commercial sources. Alternatively, sequences from AAV serotypes and strains which are published and/or available from a variety of databases may be synthesized using known techniques.

As used herein, the term “serotype” refers to an AAV which is identified by and distinguished from other AAVs based on capsid protein reactivity with defined antisera. There are at least twelve known serotypes of human AAV, including AAV1 through AAV12, however additional serotypes continue to be discovered, and use of newly discovered serotypes are contemplated. For example, AAV2 serotype is used to refer to an AAV which contains capsid proteins encoded from the cap gene of AAV2 and a genome containing 5′ and 3′ inverted terminal repeat (ITR) sequences from the same AAV2 serotype.

A “pseudotyped” AAV refers to an AAV that contains capsid proteins from one serotype and a viral genome including 5′ and 3′ inverted terminal repeats (ITRs) of a different or heterologous serotype. A pseudotyped rAAV would be expected to have cell surface binding properties of the capsid serotype and genetic properties consistent with the ITR serotype. A pseudotype rAAV may comprise AAV capsid proteins, including VP1, VP2, and VP3 capsid proteins, and ITRs from any serotype AAV, including any primate AAV serotype from AAV1 through AAV12, as long as the capsid protein is of a serotype heterologous to the serotype(s) of the ITRs. In a pseudotype rAAV, the 5′ and 3′ ITRs may be identical or heterologous. Pseudotyped rAAV are produced using standard techniques described in the art.

A “chimeric” rAAV vector encompasses an AAV vector comprising heterologous capsid proteins; that is, a rAAV vector may be chimeric with respect to its capsid proteins VP1, VP2 and VP3, such that VP1, VP2 and VP3 are not all of the same serotype AAV. A chimeric AAV as used herein encompasses AAV wherein the capsid proteins VP1, VP2 and VP3 differ in serotypes, including for example but not limited to capsid proteins from AAV1 and AAV2; are mixtures of other parvo virus capsid proteins or comprise other virus proteins or other proteins, such as for example, proteins that target delivery of the AAV to desired cells or tissues. A chimeric rAAV as used herein also encompasses a rAAV comprising chimeric 5′ and 3′ ITRs. The present invention encompasses chimeric rAAV vectors that comprise ITRs from different AAV serotypes, for example AAV1 and AAV2, or a chimeric rAAV may comprise synthetic sequences.

rAAV viral vectors may be produced by any of a number of methods known in the art including transient transfection strategies as described in U.S. Pat. Nos. 6,001,650 and 6,258,595, which are herein incorporated by reference. Typically, rAAV vector production requires four common elements: 1) a permissive host cell for replication which includes standard host cells known in the art including 293-A, 293-S (obtained from BioReliance), VERO, and HeLa cell lines which are applicable for the vector production systems described herein; 2) helper virus function which is supplied as a plasmid, pAd Helper 4.1 expressing the E2a, E4-orf6 and VA genes of adenovirus type 5 (Ad5) when utilized in transduction production systems; 3) a transpackaging rep-cap construct; and 4) a gene of interest flanked by AAV ITR sequences. Transfection production may be performed as described in the article by Sandalon et al., J. Virology, 2004; 78(22):12355-12365, herein incorporated by reference.

### Therapeutic Methods and Compositions

Some embodiments of the methods and compositions provided herein include therapeutic methods and agent to treat, inhibit or ameliorate a muscular dystrophy in a subject. Aspects useful with certain embodiments provided herein are disclosed in U.S. Pat. No. 8,221,738 and U.S. Pat. App. Pub. No. 2017/0252462 which are each incorporated by reference in its entirety, including for therapeutic compositions and methods such as kits, and routes/methods of delivery. In some embodiments, skeletal muscles comprise the muscular dystrophy. For example, non-cardiac muscles comprise the muscular dystrophy. In some embodiments, the muscular dystrophy comprises Duchenne muscular dystrophy (DMD), Becker muscular dystrophy (BMD), facioscapulohumeral muscular dystrophy, limb-girdle muscular dystrophy, or myotonic dystrophy. In some embodiments, the muscular dystrophy comprises a mutation in dystrophin gene. In some embodiments, the muscular dystrophy comprises a systemic dystrophin deficiency in a subject. For example, the level of dystrophin expressed or function of the dystrophin expressed in a skeletal muscle of the subject having the muscular dystrophy is reduced compared to the level of dystrophin expressed or function of the dystrophin expressed in a skeletal muscle of a subject not having the muscular dystrophy. In some embodiments, the muscular dystrophy is selected from DMD and BMD. In some embodiments the muscular dystrophy comprises DMD. In some embodiments the muscular dystrophy comprises myocardial remodeling and/or fibrosis.

Some embodiments include administering to the subject a polynucleotide comprising a nucleic acid encoding a sarcoplasmic/endoplasmic reticulum calcium ATPase (SERCA) polypeptide. In some embodiments, the SERCA polypeptide comprises a SERCA2 polypeptide. In some embodiments, the SERCA2 polypeptide comprises a SERCA2 isoform selected from a SERCA2a polypeptide, or a SERCA2c polypeptide. In some embodiments, the SERCA2 polypeptide comprises a SERCA2a polypeptide.

In some embodiments, the polynucleotide comprises a promoter operably linked to the nucleic acid encoding the SERCA polypeptide. In some embodiments, the promoter comprises a constitutive promoter. In some embodiments, the promoter comprises a cytomegalovirus (CMV) promoter.

In some embodiments, the polynucleotide comprises a vector. In some embodiments, the vector is selected from an adeno-associated virus (AAV) vector, a lentivirus vector, and a retrovirus vector. In some embodiments, the vector comprises an AAV vector. In some embodiments, a viral capsid comprises the polynucleotide. In some embodiments, the polynucleotide comprises a nucleic acid encoding the viral capsid. In some embodiments, the AAV vector encodes an AAV or fragment thereof having a serotype. In some embodiments, the AAV or fragment thereof comprise a viral capsid protein or fragment thereof. In some embodiments, the AAV or fragment thereof has a serotype selected from AAV serotype-1 (AAV1), AAV serotype-2 (AAV2), AAV serotype-3 (AAV3), AAV serotype-4 (AAV4), AAV serotype-5 (AAV5), AAV serotype-6 (AAV6), AAV serotype-7 (AAV7), AAV serotype-8 (AAV8), AAV serotype-9 (AAV9), AAV serotype-10 (AAVrh10), AAV serotype-11 (AAV11), and AAV serotype-12 (AAV12). In some embodiments, the AAV or fragment thereof has a serotype selected from AAV1, and AAV9. In some embodiments, the AAV or fragment thereof has an AAV1 serotype. In some embodiments, the AAV or fragment thereof has an AAV9 serotype. In some embodiments, the AAV or fragment thereof can comprise a hybrid serotype of one or more of the foregoing serotypes.

Some embodiments also include determining the presence or absence in the subject of an antibody against an AAV serotype. In some embodiments, an AAV or fragment thereof encoded by the polynucleotide can be selected to have a serotype different from the AAV serotype recognized by an antibody identified in the subject.

In some embodiments, the polynucleotide administering the polynucleotide comprises systemic administration. In some embodiments, the administering the polynucleotide comprises an injection, an infusion, or an implantation. In some embodiments, the administering the polynucleotide comprises intravenous administration. In some embodiments, the administering the polynucleotide comprises in utero administration. For example, in utero administration to an unborn subject predicted to develop the muscular dystrophy.

In some embodiments, the administering a polynucleotide comprises one or more doses of the polynucleotide administered to the subject.

In some embodiments, the administering a polynucleotide consists of a single dose of the polynucleotide administered to the subject. In some embodiments, the administering a polynucleotide consists of two doses of the polynucleotide administered to the subject. In some embodiments, the administering a polynucleotide consists of three doses of the polynucleotide administered to the subject. In some embodiments, the administering a polynucleotide consists of four doses of the polynucleotide administered to the subject. In some embodiments, the administering a polynucleotide consists of five doses of the polynucleotide administered to the subject.

In some embodiments, the polynucleotide comprises a viral vector. In some embodiments, a viral capsid or functional fragment thereof comprises the polynucleotide. In some such embodiments, a dose of the polynucleotide can be measured as a number of viral genome particles (vg), or as a number of DNase resistant particles (DRP).

In some embodiments, a dose of the polynucleotide is at least 1×10⁸ vg, 1×10⁹ vg, 1×10¹⁰ vg, 1×10¹¹ vg, 1×10¹² vg, 1×10¹³ vg, 1×10¹⁴ vg, 1×10¹⁵ vg, 1×10¹⁶ vg, 1×10¹⁷ vg, 1×10¹⁸ vg, 1×10¹⁹ vg, 1×10²⁰ vg, or an amount between any two of the foregoing amounts. In some embodiments, a dose of the polynucleotide is at least 1×10⁸ DRP, 1×10⁹ DRP, 1×10¹⁰ DRP, 1×10¹¹ DRP, 1×10¹² DRP, 1×10¹³ DRP, 1×10¹⁴ DRP, 1×10¹⁵ DRP, 1×10¹⁶ DRP, 1×10¹⁷ DRP, 1×10¹⁸ DRP, 1×10¹⁹ DRP, 1×10²⁰ DRP, or an amount between any two of the foregoing amounts. In some embodiments, a dose of the polynucleotide is within a range from about 1×10⁸ vg to about 1×10²⁰ vg, from about 1×10⁹ vg to about 1×10¹⁷ vg, from about 1×10¹⁰ vg to about 1×10¹⁶ vg, from about 1×10¹¹ vg to about 1×10¹⁵ vg, or from about 1×10¹² vg to about 1×10¹⁴ vg. In some embodiments, a dose of the polynucleotide is within a range from about 1×10¹³ vg to about 9×10¹³ vg. In some embodiments, a dose of the polynucleotide is within a range from about 1×10⁸ DRP to about 1×10²⁰ DRP, from about 1×10⁹ DRP to about 1×10¹⁷ DRP, from about 1×10¹⁰ DRP to about 1×10¹⁶ DRP, from about 1×10¹¹ DRP to about 1×10¹⁵ DRP, or from about 1×10¹² DRP to about 1×10¹⁴ DRP. In some embodiments, a dose of the polynucleotide is within a range from about 1×10¹³ DRP to about 9×10¹³ DRP.

Some embodiments also include administering a vasodilator. In some embodiments, the vasodilator is administered prior to administering the polynucleotide. In some embodiments, the vasodilator is administered concurrently with administering the polynucleotide. In some embodiments, the vasodilator comprises nitroglycerin.

In some embodiments, the subject is mammalian, such as human. In some embodiments, the subject is male. In some embodiments, the subject is an infant. As used herein, “infant” can include a human child less than 4 years old. In some embodiments, the subject is neonate. As used herein, “neonate” can include an infant human less than one month after birth. In some embodiments, the subject is in utero. In some embodiments, the subject has no substantial physical manifestation of a muscular dystrophy. For example, a subject may be predicted to develop a muscular dystrophy, such as a subject having a mutation in a gene, such as a dystrophin gene. In some embodiments, the subject lacks a physical manifestation of a muscular dystrophy, such as elevated creatinine kinase, calf pseudohypertrophy, or Gowers' sign showing weakness of the proximal muscles. In some embodiments, the polynucleotide is administered prior to an onset of muscle tissue damage. In some embodiments, the muscle tissue damage is predicted to result from the skeletal muscular dystrophy. In some embodiments, the muscle tissue damage can, or cannot, be measured by muscle histology. In some embodiments, the subject has an age of at least or not more than 1 months, 2 months, 3 months, 4 months, 5 months, 6 months, 7 months, 8 months, 9 months, 10 months, 11 months, 1 years, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, or 20 years, or a range of any two of the preceding ages, for example 1 month to 20 years, 1 month to 15 years, 1 month to 12 years, 1 month to 10 years, 3 to 20 years, 3 to 15 years, 3 to 10 years, 8 to 20 years, or 8 to 15 years.

In some embodiments, the treatment provides long-term improvement in a treated subject in one or more symptoms or measures of skeletal muscular dystrophy, such as DMD, compared to the one or more symptoms in an untreated subject having the skeletal muscular dystrophy. In some embodiments the improvement is observable for at least a period of at least 3, 6, 9, or 12 months following administration of the treatment. In some embodiments the improvement is observable for at least a period of at least 1, 2, 3, 4, 5, 6, 7, 8, 9 or 10 years following administration of the treatment. In some embodiments the improvement is in one or more measures of cardiac function or tissue. In some embodiments the improvement is in one or more measures of skeletal muscle function or tissue. In some embodiments the improvement is in one or more measures of cardiac and skeletal muscle function or tissue. Improvements include those described herein, including measures described in the examples below. For example, improvements in cardiac and/or skeletal muscle function or tissue include one or more of the following: change from baseline in left ventricular structure and function as assessed by late gadolinium enhancement (LGE) cardiac MRI including left ventricular ejection fraction, end-diastolic volume, end-systolic volume, stroke volume and/or circumferential strain; regional wall thickness; left ventricular LGE expressed as a percent of left ventricular mass and in grams; left ventricular viable mass expressed in grams; and number of left ventricular segments with LGE; composite outcome in change from baseline in LV function (LVESV), PUL 2.0, pulmonary function (pick a parameter), quality of life and terminal events; change from baseline in the following: (a) skeletal muscle function as assessed by PUL 2.0, grip strength, key and tip-to-tip pinch strength, elbow flexion strength and, if ambulatory, 10-Meter Walk/Run Time (10MWRT), incidence of loss of ambulation defined as 10MWRT>30 seconds and North Star Ambulatory Assessment (NSAA); (b) pulmonary function as assessed by slow vital capacity (SVC), forced expiratory volume in one second (FEV1), forced vital capacity (FVC), peak expiratory flow (PEF), maximum inspiratory pressure (MIP), maximum expiratory pressure (MEP), peak cough flow (PCF) and inspiratory flow reserve (IFR); and (c) quality of life as assessed by DMD UL-PROM and PODCI.

Some embodiments include methods of treating, inhibiting or ameliorating a skeletal muscular dystrophy, such as DMD in a subject, such as a human male subject, prior to an onset of muscle tissue damage predicted to result from the skeletal muscular dystrophy, the method comprising intravenous administration of a single dose comprising about 3×10¹³ vg AAV1 vector comprising a polynucleotide comprising a CMV promoter operably linked to a nucleic acid encoding a SERCA2a polypeptide. Some embodiments also include co-administration of a vasodilator, such as nitroglycerin.

Some embodiments include methods of treating, inhibiting or ameliorating a skeletal muscular dystrophy, such as DMD in a subject, such as a human male subject, prior to an onset of muscle tissue damage predicted to result from the skeletal muscular dystrophy, the method comprising administration by intracoronary infusion of a single dose comprising about 3×10¹³ vg AAV1 vector comprising a polynucleotide comprising a CMV promoter operably linked to a nucleic acid encoding a SERCA2a polypeptide. Some embodiments also include co-administration of a vasodilator, such as nitroglycerin.

Some embodiments include methods of treating, inhibiting or ameliorating a skeletal muscular dystrophy, such as DMD in a subject, such as a human male subject, prior to an onset of muscle tissue damage predicted to result from the skeletal muscular dystrophy, the method comprising an intracoronary infusion of a single dose comprising about 3×10¹³ vg AAV1 vector comprising a polynucleotide comprising a CMV promoter operably linked to a nucleic acid encoding a SERCA2a polypeptide administered into the left and/or right coronary artery via antegrade epicardial coronary artery infusion with commercially available guide or diagnostic cardiac catheters and the B. Braun Perfusor® Space Syringe Pump set at a flow rate of 300 mL/hr over 10 minutes. In some embodiments, a nitroglycerin IV infusion is administered for a minimum of 10 minutes at the highest tolerated dose prior to infusion and concomitantly with the infusion of the AAV1 vector.

### Screening for Therapeutic Agents

Some embodiments of the methods and compositions provided herein include screening for a therapeutic agent to treat, inhibit or ameliorate a skeletal muscular dystrophy in a patient. In some embodiments, the skeletal muscular dystrophy comprises a systemic dystrophin deficiency in the patient. In some embodiments, the skeletal muscular dystrophy is selected from DMD and BMD. In some embodiments, the muscular dystrophy comprises DMD. In some embodiments the muscular dystrophy comprises myocardial remodeling and/or fibrosis.

Some such embodiments include (a) contacting a test agent with a ventricular cardiac tissue strip; (b) measuring a contractile amplitude of the ventricular cardiac tissue strip contacted with the test agent; (c) comparing the contractile amplitude of the ventricular cardiac tissue strip contacted with the test agent with a contractile amplitude of a ventricular cardiac tissue strip not contacted with the test agent; and (d) determining that the test agent comprises a therapeutic agent based on the comparison.

In some embodiments, the ventricular cardiac tissue strip is obtained by: (i) differentiating a population of induced pluripotent stem cells to obtain a plurality of cardiospheres comprising a plurality of the ventricular cardiomyocytes; (ii) dissociating the plurality of cardiospheres to obtain a plurality of cardiac cells; and (iii) contacting the plurality of cardiac cells with a population of fibroblasts in the presence of collagen under conditions to obtain the ventricular cardiac tissue strip. Some embodiments also include obtaining the ventricular cardiac tissue strip, comprising: (i) differentiating a population of induced pluripotent stem cells to obtain a plurality of cardiospheres comprising a plurality of the ventricular cardiomyocytes; (ii) dissociating the plurality of cardiospheres to obtain a plurality of cardiac cells; and (iii) contacting the plurality of cardiac cells with a population of fibroblasts in the presence of collagen under conditions to obtain the ventricular cardiac tissue strip. In some embodiments, the population of induced pluripotent stem cells is obtained from a subject having a muscular dystrophy, such as DMD or BMD.

In some embodiments, the test agent is contacted with the ventricular cardiac tissue strip for a period of at least 1 hour, 2, hours, 3 hours, 4 hours, 5 hours, 10 hours, 12 hours, 18 hours, 24 hours, or for a period between any two of the foregoing numbers. In some embodiments, the test agent is contacted with the ventricular cardiac tissue strip for a period of at least 1 day, 2 days, 3 days, 5 days, 10 days, 15 days, 20 days, 25 days, 30 days, or for a period between any two of the foregoing numbers.

In some embodiments, the ventricular cardiac tissue strip is stimulated by an electrical field during the measuring a contractile amplitude of the ventricular cardiac tissue strip contacted with the test agent. In some embodiments a frequency of the electrical filed can be constant during the measuring. In some embodiments a frequency of the electrical filed can be modulated during the measuring. In some embodiments, a parameter can be determined during the measuring. In some embodiments, the parameter is selected from developed force, normalized developed force, rate variability, force variability, force-frequency relationship, and beta adrenergic response.

In some embodiments, the test agent comprises a polynucleotide. In some embodiments, the polynucleotide encodes a SERCA polypeptide. In some embodiments, the SERCA polypeptide comprises a SERCA2 polypeptide. In some embodiments, the SERCA2 polypeptide comprises a SERCA2 isoform selected from a SERCA2a polypeptide, or a SERCA2c polypeptide. In some embodiments, the SERCA2 polypeptide comprises a SERCA2a polypeptide.

In some embodiments, the polynucleotide comprises a promoter operably linked to the nucleic acid encoding the SERCA polypeptide. In some embodiments, the promoter comprises a constitutive promoter. In some embodiments, the promoter comprises a CMV promoter. In some embodiments, the promoter comprises an inducible promoter.

In some embodiments, the polynucleotide comprises a vector. In some embodiments, the vector is selected from an AAV vector, a lentivirus vector, and a retrovirus vector. In some embodiments, the vector comprises an AAV vector. In some embodiments, a viral capsid comprises the polynucleotide. In some embodiments, the polynucleotide comprises a nucleic acid encoding the viral capsid. In some embodiments, the AAV vector encodes an AAV or fragment thereof having a serotype. In some embodiments, the AAV or fragment thereof comprise a viral capsid protein or fragment thereof. In some embodiments, the AAV or fragment thereof has a serotype selected from AAV1, AAV2, AAV3, AAV4, AAV5, AAV6, AAV7, AAV8, AAV9, AAV10, AAV11, and AAV12. In some embodiments, the AAV or fragment thereof has a serotype selected from AAV1, and AAV9. In some embodiments, the AAV or fragment thereof has an AAV1 serotype. In some embodiments, the AAV or fragment thereof has an AAV9 serotype. In some embodiments, the AAV or fragment thereof can comprise a hybrid serotype of one or more of the foregoing serotypes.

### Systems and Kits

Some embodiments of the methods and compositions provided herein include systems and kits to treat, inhibit or ameliorate a skeletal muscular dystrophy in a subject. Some such embodiments include a polynucleotide encoding a SERCA2 polypeptide. Some embodiments include a pharmaceutical composition comprising the polynucleotide and a pharmaceutically acceptable excipient. In some embodiments, the SERCA polypeptide comprises a SERCA2 polypeptide. In some embodiments, the SERCA2 polypeptide comprises a SERCA2 isoform selected from a SERCA2a polypeptide, or a SERCA2c polypeptide. In some embodiments, the SERCA2 polypeptide comprises a SERCA2a polypeptide. In some embodiments, the polynucleotide comprises a promoter operably linked to the nucleic acid encoding the SERCA polypeptide. In some embodiments, the promoter comprises a constitutive promoter. In some embodiments, the promoter comprises a CMV promoter. In some embodiments, the polynucleotide comprises a vector. In some embodiments, the vector is selected from an AAV vector, a lentivirus vector, and a retrovirus vector. In some embodiments, the vector comprises an AAV vector. In some embodiments, a viral capsid comprises the polynucleotide. In some embodiments, the polynucleotide comprises a nucleic acid encoding the viral capsid. In some embodiments, the AAV vector encodes an AAV or fragment thereof having a serotype. In some embodiments, the AAV or fragment thereof comprise a viral capsid protein or fragment thereof. In some embodiments, the AAV or fragment thereof has a serotype selected from AAV1, AAV2, AAV3, AAV4, AAV5, AAV6, AAV7, AAV8, AAV9, AAV10, AAV11, and AAV12. In some embodiments, the AAV or fragment thereof has a serotype selected from AAV1, and AAV9. In some embodiments, the AAV or fragment thereof has an AAV1 serotype. In some embodiments, the AAV or fragment thereof has an AAV9 serotype.

More embodiments include a container, such as a sterile vial, comprising a single dose of the polynucleotide to treat, inhibit or ameliorate a skeletal muscular dystrophy in a subject. In some embodiments, the polynucleotide comprises a viral vector. In some embodiments, a viral capsid or functional fragment thereof comprises the polynucleotide. In some such embodiments, a dose of the polynucleotide can be measured as a number of viral genome particles (vg), or as a number of DRP.

In some embodiments, a dose of the polynucleotide is at least 1×10⁸ vg, 1×10⁹ vg, 1×10¹⁰ vg, 1×10¹¹ vg, 1×10¹² vg, 1×10¹³ vg, 1×10¹⁴ vg, 1×10¹⁵ vg, 1×10¹⁶ vg, 1×10¹⁷ vg, 1×10¹⁸ vg, 1×10¹⁹ vg, 1×10²⁰ vg, or an amount between any two of the foregoing amounts. In some embodiments, a dose of the polynucleotide is at least 1×10⁸ DRP, 1×10⁹ DRP, 1×10¹⁰ DRP, 1×10¹¹ DRP, 1×10¹² DRP, 1×10¹³ DRP, 1×10¹⁴ DRP, 1×10¹⁵ DRP, 1×10¹⁶ DRP, 1×10¹⁷ DRP, 1×10¹⁸ DRP, 1×10¹⁹ DRP, 1×10²⁰ DRP, or an amount between any two of the foregoing amounts. In some embodiments, a dose of the polynucleotide is within a range from about 1×10⁸ vg to about 1×10²⁰ vg, from about 1×10⁹ vg to about 1×10¹⁷ vg, from about 1×10¹⁰ vg to about 1×10¹⁶ vg, from about 1×10¹¹ vg to about 1×10¹⁵ vg, or from about 1×10¹² vg to about 1×10¹⁴ vg. In some embodiments, a dose of the polynucleotide is within a range from about 1×10¹³ vg to about 9×10¹³ vg. In some embodiments, a dose of the polynucleotide is within a range from about 1×10⁸ DRP to about 1×10²⁰ DRP, from about 1×10⁹ DRP to about 1×10¹⁷ DRP, from about 1×10¹⁰ DRP to about 1×10¹⁶ DRP, from about 1×10¹¹ DRP to about 1×10¹⁵ DRP, or from about 1×10¹² DRP to about 1×10¹⁴ DRP. In some embodiments, a dose of the polynucleotide is within a range from about 1×10¹³ DRP to about 9×10¹³ DRP.

Some embodiments also include a vasodilator, such as nitroglycerin.

### CERTAIN EMBODIMENTS

Some embodiments of the methods and compositions provided herein include the use of a vasodilator. Aspects useful with certain embodiments provided herein are disclosed in U.S. Pat. No. 8,221,738 which is incorporated by reference in its entirety, and for compositions, kits, and routes/methods of delivery. In some embodiments, the polynucleotide is administered in combination with a vasodilator. In some embodiments, a vasodilator can be administered to a subject prior to administration of the polynucleotide. In some embodiments, a vasodilator can be administered to a subject prior to and concurrently with administration of the polynucleotide. Examples of vasodilators include adenosine, histamine (or histamine-inducing agents), alpha blockers, theobromine, papaverine, ethanol, tetrahydrocannabinol (THC), minoxidil, nitric oxide (including nitric oxide increasing substances), and nitroglycerin. In some embodiments, a vasodilator is administered systemically, for example orally, transdermally, or by intravenous injection or infusion. In some embodiments, the infusion comprises an intracoronary infusion.

### EXAMPLES

### Example 1-In Vivo Administration of SERCA2a in a DMD Model

### A Single Intravenous Injection of an AAV9 SERCA2a Vector to Young Mdx Mice Resulted in Improved SR Calcium Uptake at 21 Months of Age

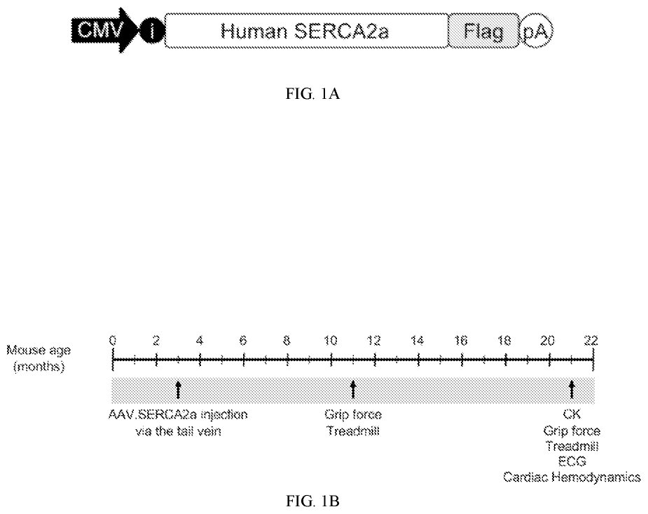

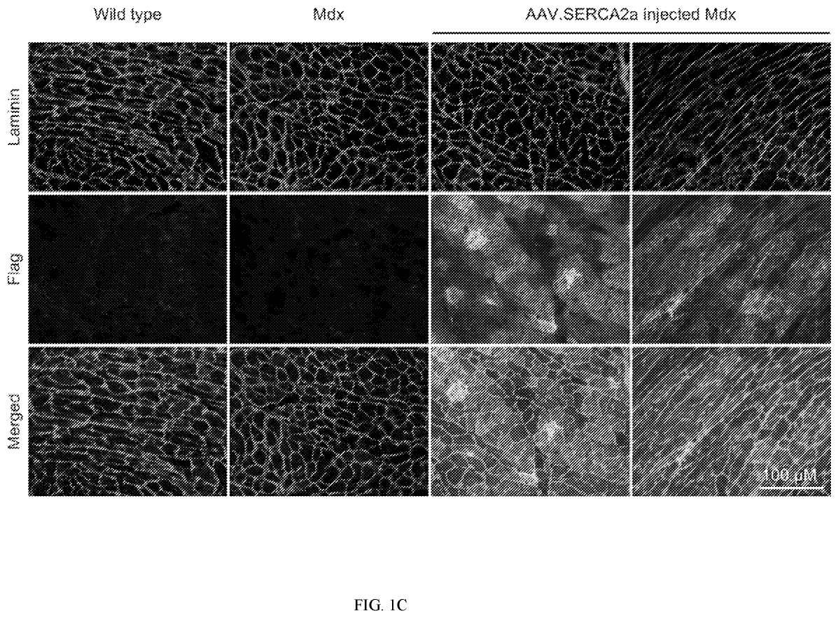

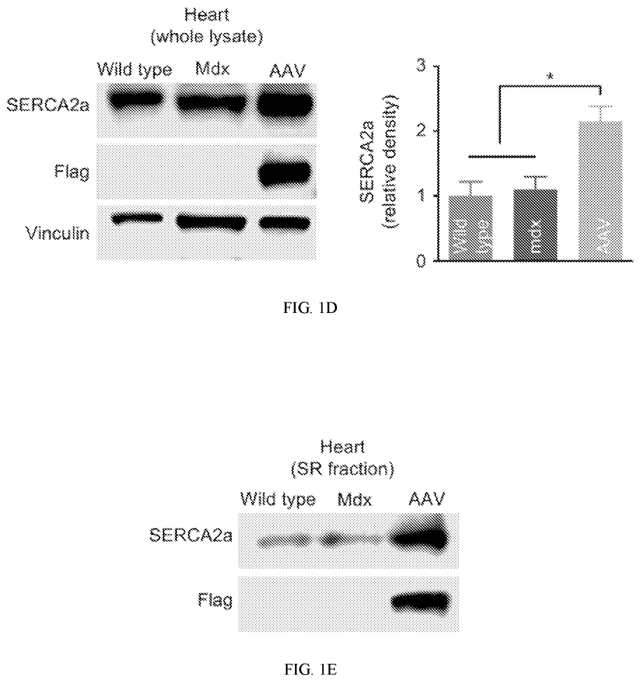

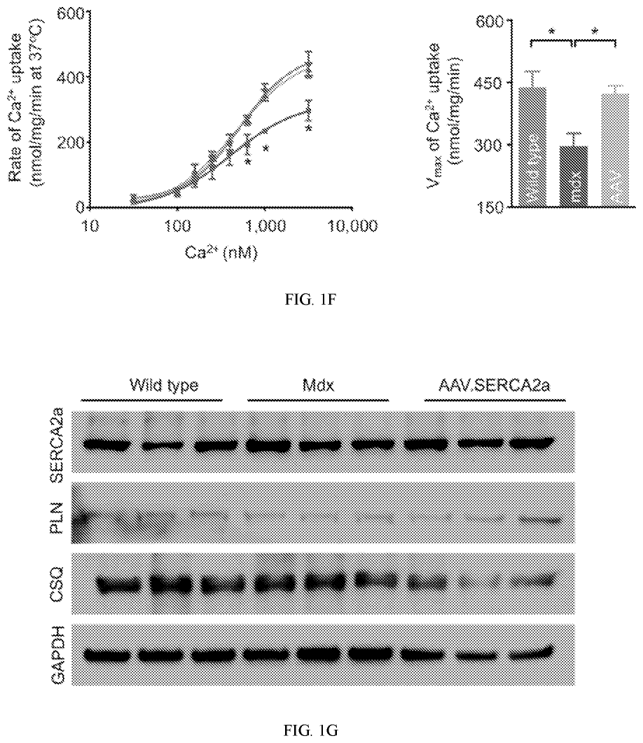

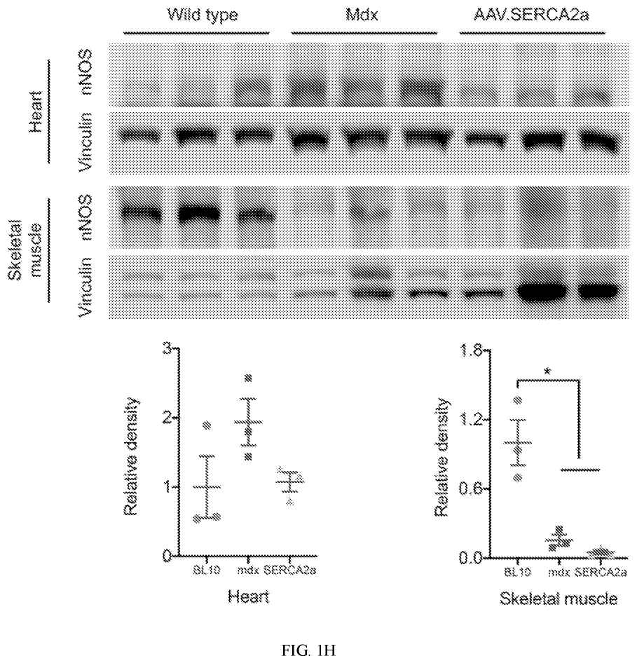

To test SERCA2a as a disease-modifying gene therapy for DMD, a flag-tagged human SERCA2a gene was packaged in AAV9 and administered to 3-month old mdx mice via the tail vein at the dose of 6×10¹² viral genome (vg) particles/mouse (FIG. TA, FIG. 1B). The average lifespan of mdx mice is about 21.5 months.^{11,12} Hence, a terminal function assay was performed and heart and skeletal muscle was harvested when treated mice reached 21 months of age.

Immunofluorescence staining with the flag antibody showed robust cardiac expression of human SERCA2a in treated mice (FIG. 1C). Total cell lysate western blot revealed a significant increase of the total SERCA2a level in the heart (FIG. 1D, FIG. 1G, FIG. 1H). Western blot with the heart SR preparation revealed correct localization of flag-tagged human SERCA2a and enrichment of SERCA2a in the SR of AAV-injected mice (FIG. 1E). No substantial changes were observed in the expression of other calcium regulating proteins such as phospholamban and calsequestrin (FIG. 1G). SR calcium uptake was significantly reduced in the mdx heart (FIG. 1F). This was completely normalized in the heart of AAV9-treated mdx mice (FIG. 1F).

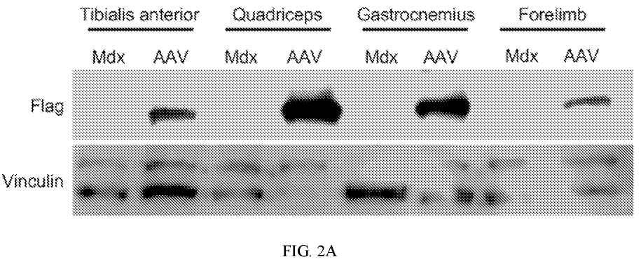

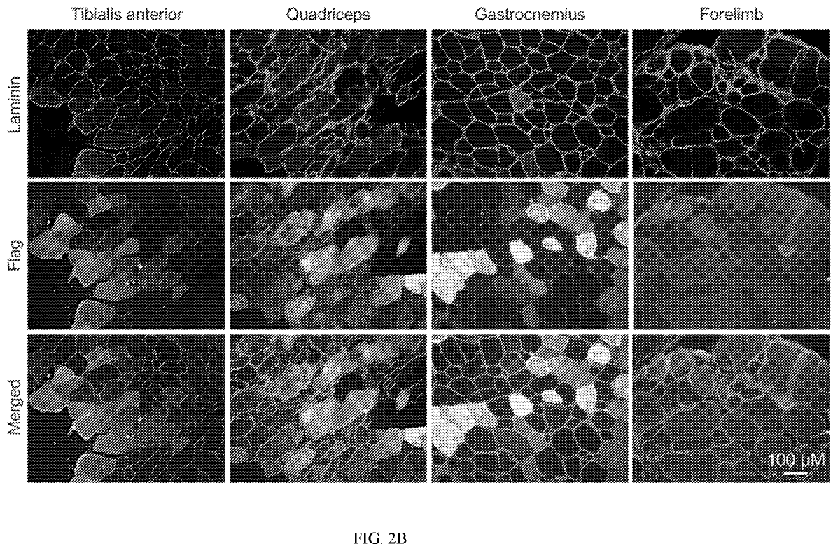

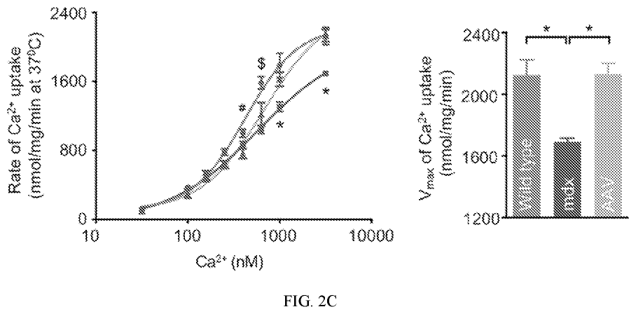

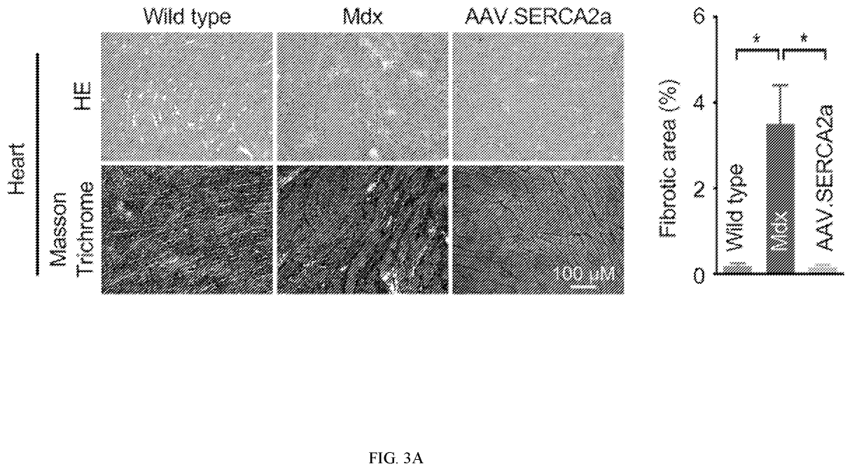

AAV9-mediated SERCA2a expression and calcium uptake in skeletal muscle was also examined (FIG. 2). Flag tag western blot and immunostaining showed widespread human SERCA2a expression in forelimb muscle, upper hindlimb muscle (quadriceps), and lower hind limb muscles (tibialis anterior and gastrocnemius) (FIG. 2A, FIG. 2B). Similar to what was found in the heart, SERCA2a therapy also significantly enhanced SR calcium uptake in skeletal muscle. The maximum rate of calcium uptake in treated mice was indistinguishable from that of normal mice (FIG. 2C).

Besides SERCA2a and other calcium regulating proteins, neuronal nitric oxide synthase (nNOS) expression was also evaluated (FIG. 1H). No see a significant difference was observed in the cardiac nNOS level between normal and mdx hearts. AAV.SERCA2a treatment did not significantly change the cardiac nNOS level either (FIG. 1H). Consistent with previous studies,^{14,15} the skeletal muscle nNOS level was significantly reduced in mdx mice compared to that of BL10 mice. The nNOS level in the skeletal muscle of AAV.SERCA2a-treated mice was not significantly different from that of untreated mdx mice (FIG. 1H).

### Systemic SERCA2a Therapy in Young Mdx Mice Prevented Dilated Cardiomyopathy in Terminal Age Mdx Mice

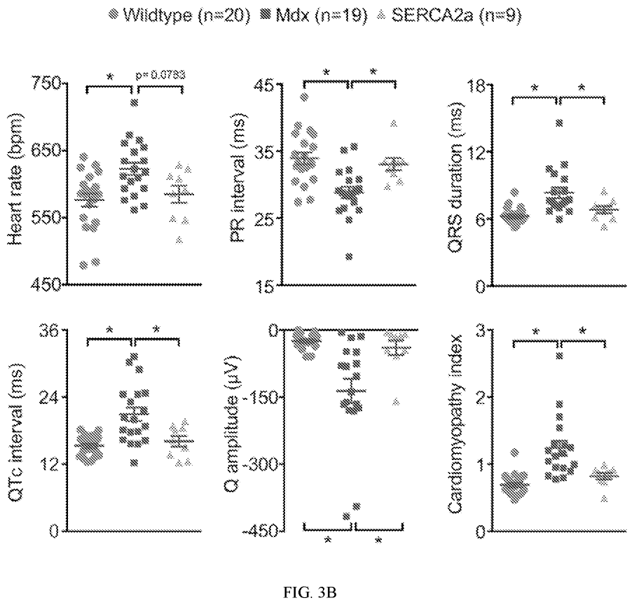

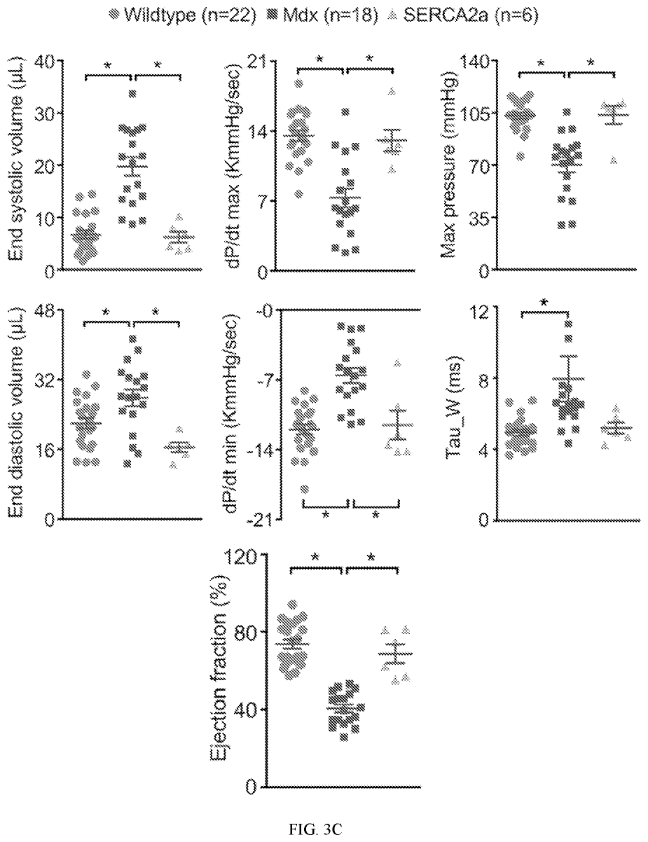

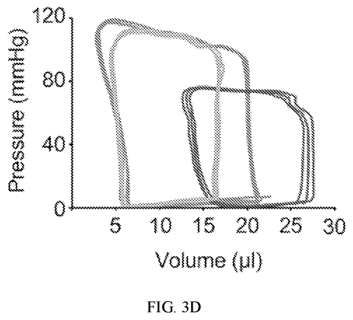

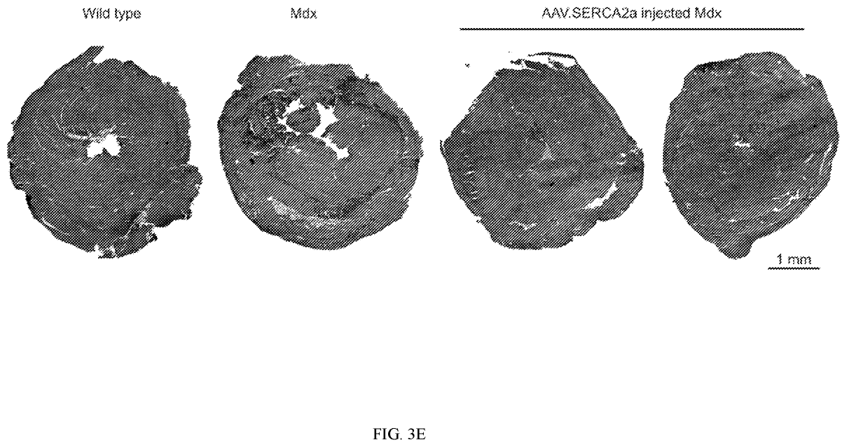

Untreated mdx mice displayed characteristic features of dilated cardiomyopathy at 21 months of age (FIGS. 3A-E).^{16,17} These include myocardial fibrosis, chamber dilation, aberrant ECG, and hemodynamic dysfunction (FIGS. 3A-3E). On cardiac catheterization, mdx mice showed a significant increase of the ventricular volume at the end of the systole and diastole, a significant decrease of heart contractility, and a rightward/downward shift of the pressure-volume loop (FIGS. 3C-3E; TABLE 1). Systemic SERCA2a injection completely prevented heart muscle fibrosis and normalized the PR interval, QRS duration, corrected QT (QTc) interval, Q amplitude, cardiomyopathy index, end-systolic volume, end-diastolic volume, maximum pressure, end-systolic pressure, maximum and minimum rates of ventricular pressure change (dP/dt max and dP/dt min), pressures at dP/dt max, volume at dP/dt max and dP/dt min preload adjusted maximum power, and ejection fraction (FIGS. 3A-3E; TABLE 1). Treatment significantly improved the stroke work (TABLE 1). A trend was observed of improvement in the heart rate, left ventricular relaxation time constant (Tau), stroke volume index, cardiac index, end-diastolic pressure, and maximum power (FIGS. 3A-D; TABLE 1). Anatomic parameters of the heart (weight and weight ratios) were unremarkable in treated mice (TABLE 2).

TABLE 1

| Parameter | wild-type | mdx | AAV.SERCA2a |
|---|---|---|---|
| Sample Size (N) | 22 | 18 | 6 |
| Body surface area (cm²) | 93.85 ± 1.83ª | 76.53 ± 6.10 | 71.86 ± 7.47 |
| Stroke volume index (mL/m²) | 1.79 ± 0.12 | 1.38 ± 0.11° | 1.70 ± 0.12 |
| Cardiac index (L/min/m²) | 1.10 ± 0.07^{b} | 0.82 ± 0.06 | 0.93 ± 0.10 |
| Min P (mmHg) | 2.95 ± 0.59 | 3.34 ± 0.83 | 1.74 ± 0.3 |
| End systolic pressure (mmHg) | 96.59 ± 2.34 | 67.62 ± 4.79ª | 96.02 ± 5.56 |
| End diastolic pressure (mmHg) | 6.42 ± 0.72 | 5.53 ± 0.95 | 6.73 ± 1.01 |
| Stroke work (KmmHgpL) | 1.37 ± 0.09ª | 0.55 ± 0.05ª | 0.99 ± 0.05ª |
| Arterial elastance (mmHg/pL) | 6.09 ± 0.47 | 6.39 ± 0.64 | 8.23 ± 0.81 |
| dV/dt max (pL/sec) | 898.4 ± 58.1^{b} | 681.4 ± 51.5 | 702.7 ± 45.4 |
| dV/dt min (pL/sec) | −1023.6 ± 63.4^{b} | −790.9 ± 58.7 | −816.0 ± 80.0 |
| P@dV/dt max (mmHg) | 29.9 ± 6.20 | 26.9 ± 5.50 | 17.4 ± 7.86 |
| P@dP/dt max (mmHg) | 65.7 ± 2.00 | 40.3 ± 3.67ª | 64.6 ± 4.00 |
| V@dP/dt max (pL) | 22.3 ± 1.17 | 27.8 ± 1.76ª | 17.5 ± 0.97 |
| V@dP/dt min (pL) | 7.11 ± 0.66 | 18.8 ± 1.54ª | 6.49 ± 0.92 |
| Max Power (mWatts) | 11.48 ± 0.82^{b} | 5.21 ± 0.38 | 8.34 ± 0.60 |
| Preload adjusted max power | 282.6 ± 31.8 | 92.9 ± 17.4ª | 332.0 ± 48.8 |
| (mWatts/pL²) | | | |

^{a}significantly different from other two groups.
^{b}significantly different from mdx.
^{c}p = 0.0725 between BL10 and mdx.
Abbreviations:
Min P, minimum pressure;
dV/dt, rate of left ventricular volume change;
dV/dt max, maximum value of dV/dt during cardiac cycle;
dV/dt min, minimum value of dV/dt during cardiac cycle;
P@dV/dt max, pressure at dV/dt max;
dP/dt, rate of left ventricular pressure change;
P@dP/dt max, pressure at the maximum value of dP/dt during cardiac cycle;
V@dP/dt max, volume at the dP/dt max;
V@dP/dt min, volume atthe dP/dt min;
Max power, maximum value of the power during cardiac cycle (Power = pressure*dV/dt)

TABLE 2

| Parameter | BL10 | mdx | AAV.SERCA2a |
|---|---|---|---|
| Sample Size (n) | 22 | 17 | 9 |
| Age (m) | 21.92 ± 0.42 | 21.16 ± 1.52 | 20.80 ± 1.75 |
| BW (g) | 29.57 ± 0.88ª | 21.78 ± 1.59 | 19.78 ± 1.89 |
| HW (mg) | 116.9 ± 1.68ª | 107.5 ± 9.67 | 102.4 ± 10.80 |
| VW (mg) | 110.5 ± 1.50ª | 100.1 ± 9.58 | 97.8 ± 10.40 |
| TL (mm) | 18.51 ± 0.07 | 18.50 ± 2.17 | 18.37 ± 1.84 |
| TW (mg) | 36.42 ± 1.03 | 37.93 ± 3.32 | 39.26 ± 3.81 |
| HW/BW (mg/g) | 4.02 ± 0.12ª | 4.94 ± 1.08 | 5.18 ± 0.90 |
| HW/TL (mg/mm) | 6.45 ± 0.1 lª | 5.68 ± 1.16 | 5.57 ± 0.58 |
| HW/TW (mg/g) | 3.27 ± 0.13^{b} | 2.95 ± 1.08 | 2.65 ± 1.01 |
| VW/BW (mg/g) | 3.80 ± 0.1 lª | 4.66 ± 1.15 | 4.95 ± 0.85 |
| VW/TL (mg/mm) | 6.09 ± 0.1 lª | 5.36 ± 1.15 | 5.33 ± 0.57 |
| VW/TW (mg/g) | 3.09 ± 0.12^{b} | 2.72 ± 1.16 | 2.54 ± 0.97 |

Abbreviations:
BW, body weight;
HW, heart weight;
TL, tibia length;
TW, tibialis muscle weight;
VW, ventricle weight
^{a}significantly different from other two groups
^{b}significantly different from SERCA2a group only

### SERCA2a Therapy in 3-Month-Old Mdx Mice Significantly Enhanced Forelimb Grip Strength and Treadmill Running at 11 and 21 Months of Age

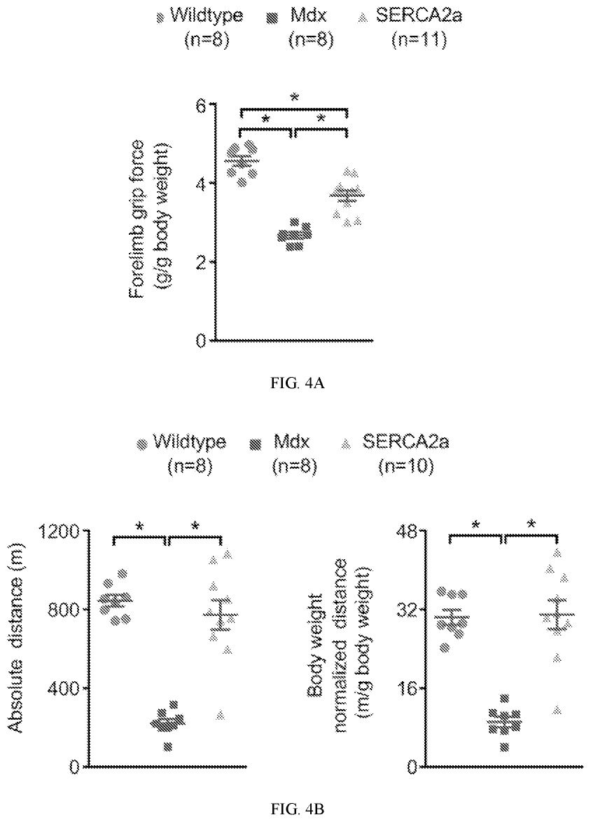

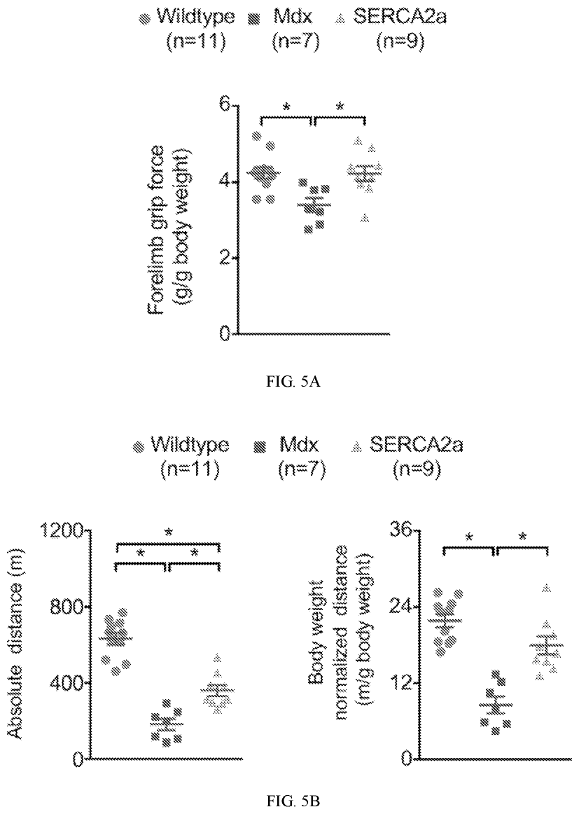

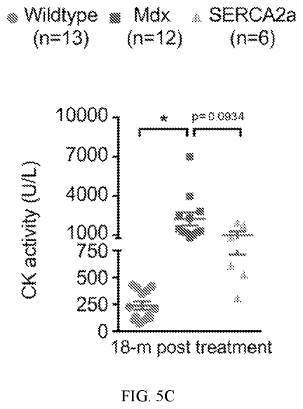

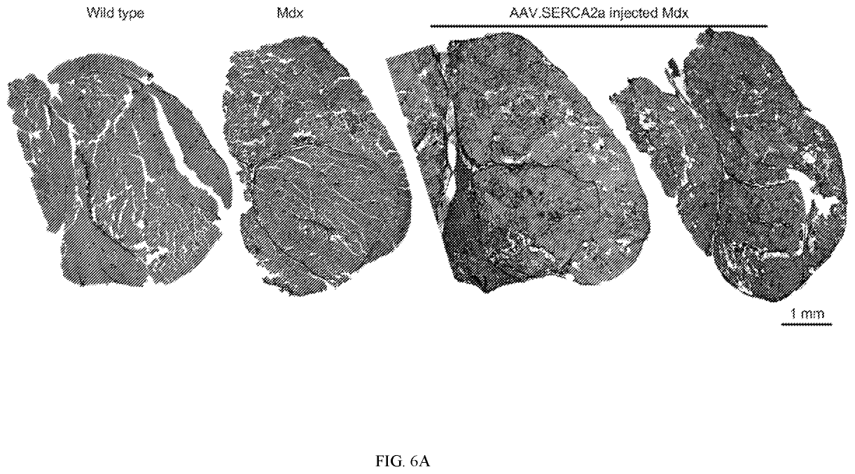

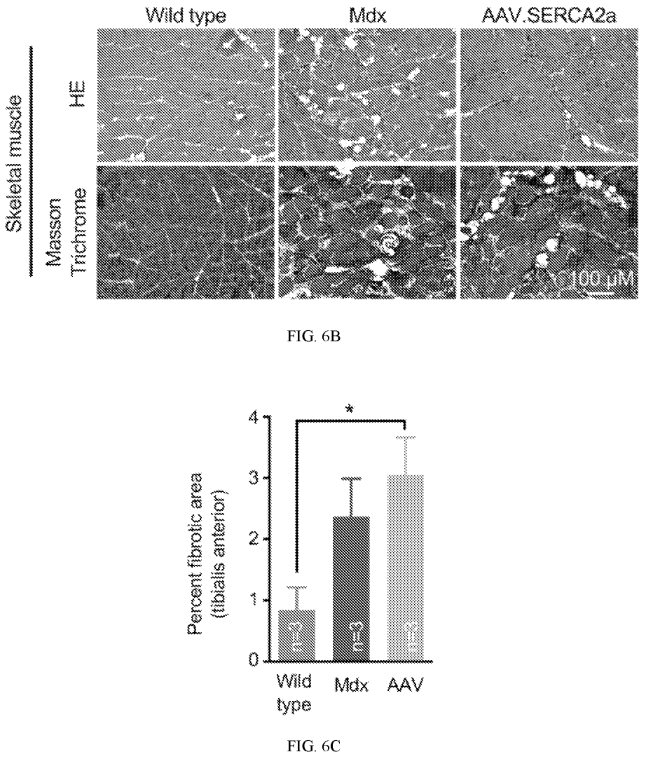

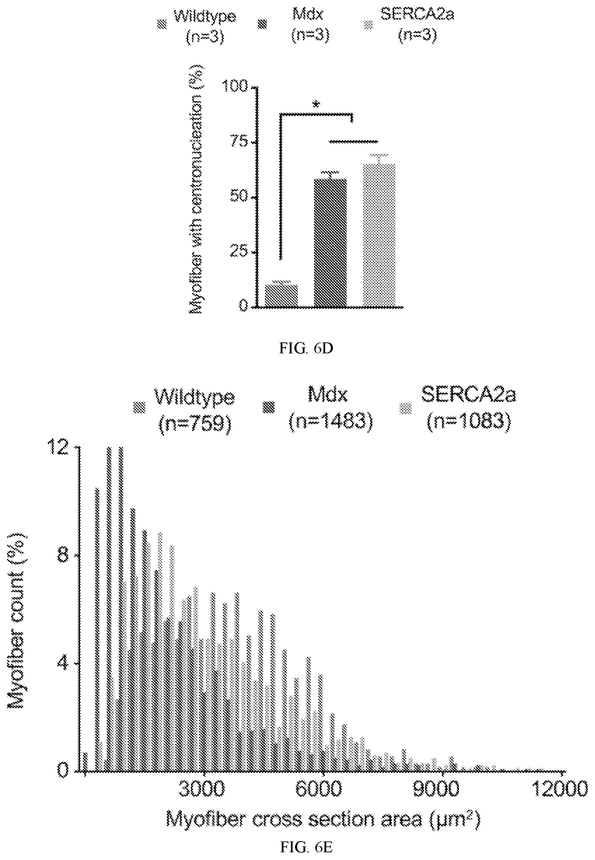

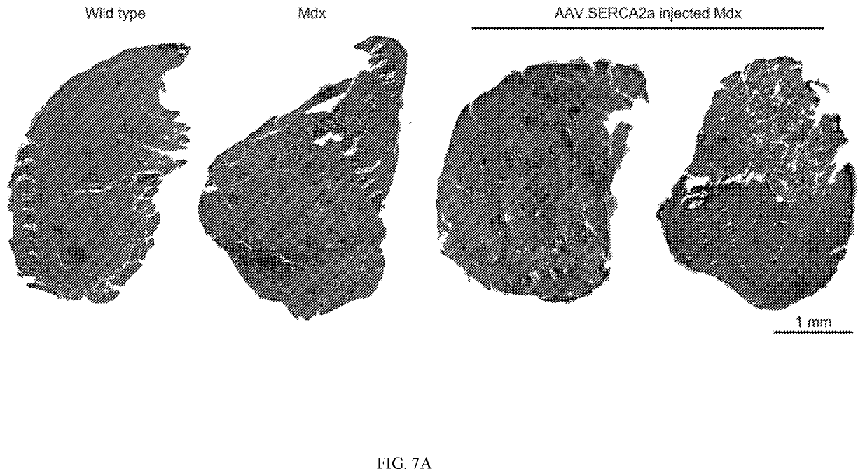

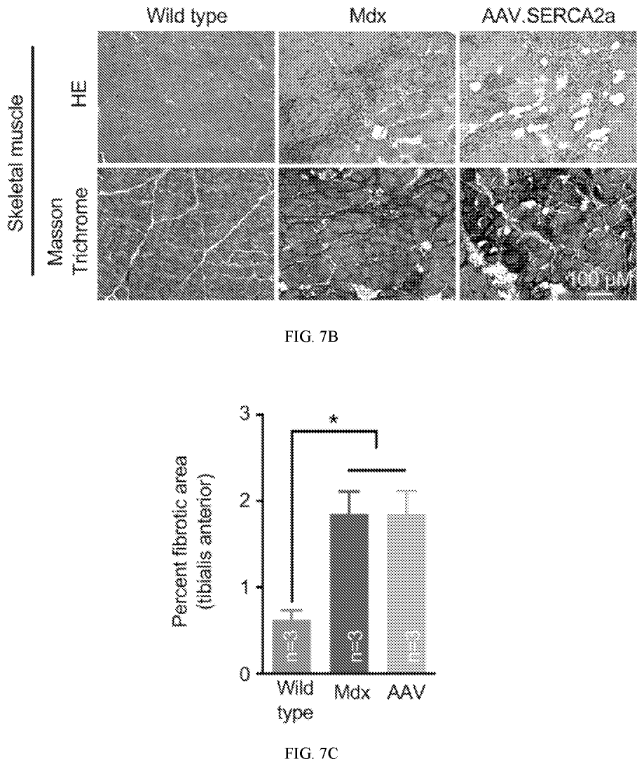

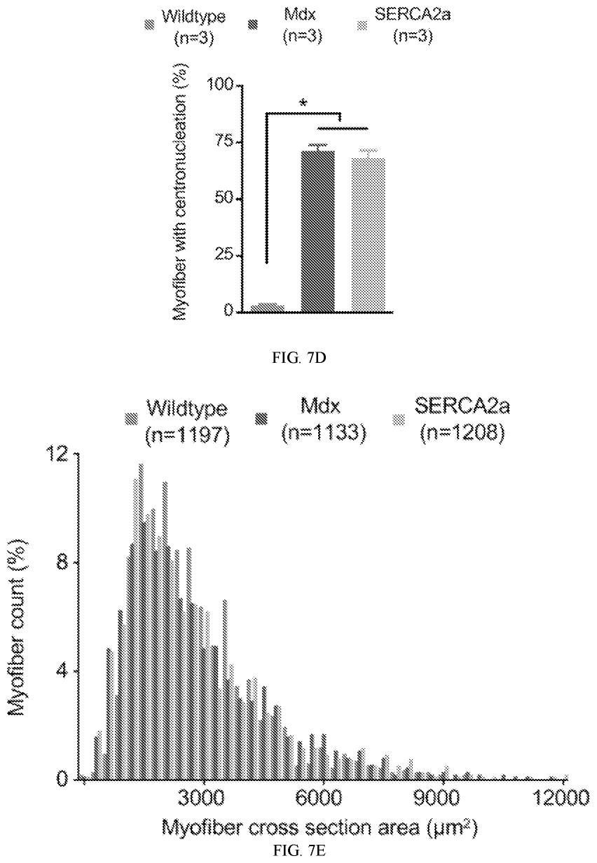

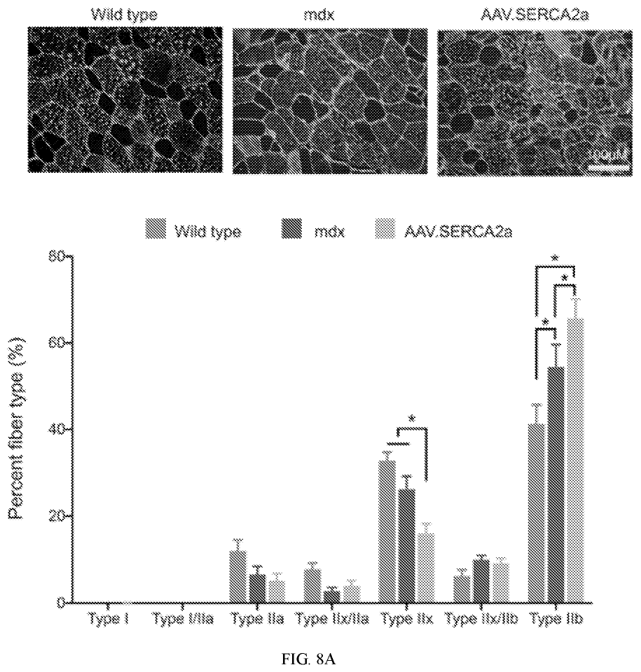

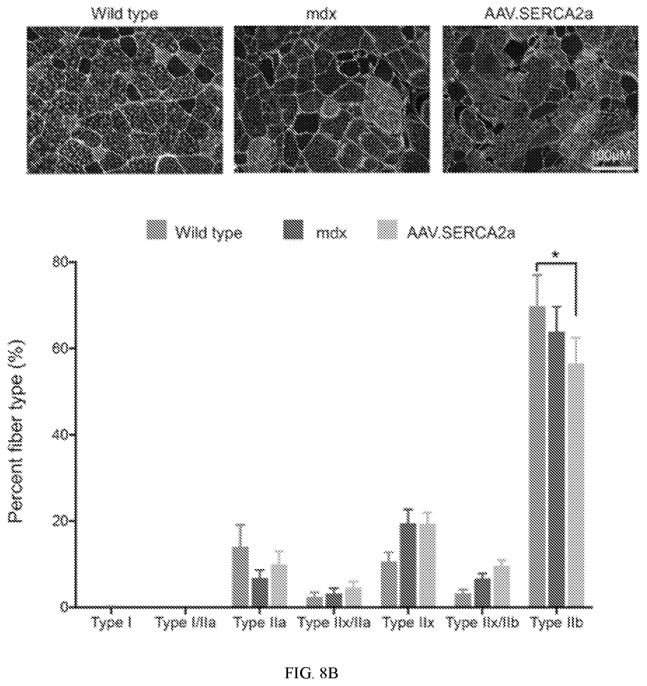

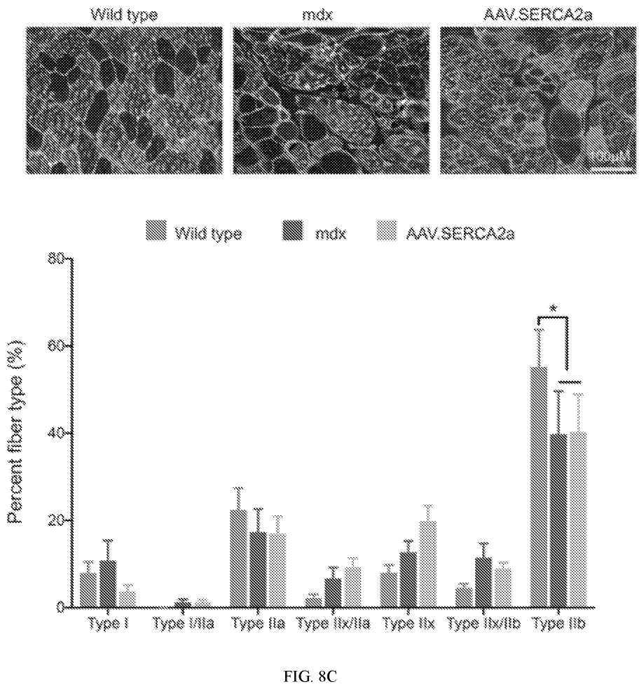

Skeletal muscle function and whole-body performance were evaluated by noninvasive grip strength measurement and treadmill running assay, respectively, at the age of 11 and 21 months (FIGS. 4A-4B, FIGS. 5A-5C). Compared with that of untreated mdx mice, SERCA2a treated mdx mice showed a significantly higher forelimb grip force (FIG. 4A and FIG. 5A). On treadmill assay, both absolute running distance and body weight-normalized running distance were restored to the wild-type levels at 11 months of age (FIG. 4B). By 21 months of age, treated mdx mice still run significantly longer than do untreated mdx mice (FIG. 5B). A trend was also detected of reduction in the serum creatine kinase (CK) level in treated mice at 21 months of age (p=0.09) (FIG. 5C). Skeletal muscle histology was evaluated by hematoxylin and eosin staining, Masson trichrome staining and myofiber type immunostaining (FIGS. 6A-6E, FIGS. 7A-7E, FIGS. 8A-8C). Despite the improvement in grip strength and running performance, nominal improvement was observed in skeletal muscle histology (FIGS. 6A-6E, FIGS. 7A-7E).

In embodiments disclosed herein, a single systemic human SERCA2a therapy resulted in lifelong improvement of muscle and heart function in the mdx mouse model for DMD. A flag-tagged human SERCA2a AAV9 vector was delivered intravenously to 3-month-old mdx mice. When mice reached 21 months of age, persistent and widespread human SERCA2a expression was observed in striated muscles throughout the body. Treatment normalized defective SR calcium uptake in the heart and skeletal muscle (FIGS. 1A-1H, FIGS. 2A-2C). On physiological assays, SERCA2a therapy significantly increased the forelimb muscle grip force and treadmill running distance (FIGS. 4A-4B, FIGS. 5A-5C). Remarkably, systemic SERCA2a therapy completely prevented myocardial fibrosis and normalized cardiac electrophysiology (FIGS. 3A-3E). Major hemodynamic parameters at the systole and diastole were restored to the wild-type level (FIGS. 3A-3E; TABLE 1). These results indicated that mechanism-based gene therapy with a disease modifier (such as using SERCA2a to restore cytosolic calcium homeostasis in DMD) was an important avenue to effectively treat at least muscular dystrophy.

DMD is caused by dystrophin deficiency. Hence, restoration of dystrophin expression has been the primary focus of experimental DMD gene therapy studies. Highly encouraging results have been achieved in murine and canine DMD models with dystrophin gene replacement and dystrophin gene repair therapies.¹⁸⁻²⁰ Several clinical trials are now ongoing with systemic AAV micro-dystrophin gene therapy.¹⁰ Despite the progress, dystrophin-based gene therapy is not without limitations. For example, newly restored dystrophin could be recognized as a new antigen by the immune system and induce an immune reaction.²¹ Mutation-targeted exon-skipping and genomic-editing approaches have to be individually tailored to the mutation. Novel therapies that utilize genes naturally expressed in DMD patients may overcome these issues.

Aberrant intracellular cytosolic calcium elevation is a primary contributor to muscle necrosis in DMD. In normal muscle, the cytosolic calcium level is maintained at the physiological level by coordinated regulations of calcium entry and recycling among the extracellular space, cytoplasm, and intracellular calcium storage organelles. In dystrophic muscle, calcium homeostasis is disrupted. More calcium enters the cytosol via dysfunctional calcium channels and the leaky ryanodine receptor (RyR).^{22,23} Less calcium is removed from the cytosol due to reduced SERCA activity.

Calcium modulation has been considered a therapeutic target for DMD since the early 1980s.²⁴ However, clinical benefits have not been observed in human trials with drugs that block calcium channels.²⁵ With the recent recognition of RyR leakage as a primary contributor to excessive cytosolic calcium entry, investigators have begun to explore RyR-stabilizing chemicals.^{22,26}

Another approach to restore calcium homeostasis is to enhance calcium uptake by SERCA. This can be achieved by modulating SERCA activity or expressing more SERCA. Abating the inhibitory SERCA regulator (such as phospholamban and sarcolipin) has been shown to suppress heart failure in a hamster model of limb girdle muscular dystrophy and to ameliorate the dystrophic phenotype in DMD mouse models.²⁷⁻²⁹ SERCA overexpression has been tested using either SERCA1a or SERCA2a. Three independent groups explored SERCA1a upregulation in murine DMD models using the transgenic approach or neonatal AAV gene transfer.³⁰⁻³² These studies showed significant improvements in calcium uptake, histology, and function of skeletal muscle. A human SERCA2a vector delivered to 12-month-old mdx mice and observed significant improvements in several ECG parameters at 20 months of age.³³ The same human SERCA2a vector was tested in the skeletal muscle of neonatal limb girdle muscular dystrophy mice and found significant reduction of muscle degeneration/regeneration. ³⁰

However, despite the foregoing outcomes, it was uncertain whether SERCA therapy could have provided long-term protection to both skeletal muscle and heart when the therapy is started at an age similar to the age targeted by ongoing DMD gene therapy trials.¹⁰ With this in mind, in the experiments disclosed herein, mdx mice were targeted at an age equivalent to that of teenage boys in humans.³⁴ Treated mice were followed until the end of their expected lifespan and evaluated both skeletal muscle and cardiac outcomes. The long-term follow up of all striated muscles is important because an effective DMD therapy requires lifelong protection of both skeletal and cardiac muscle.³⁵ In addition, a model was used that fully recapitulates dilated cardiomyopathy in DMD patients.³⁶ With improved medical care and ventilation support, more patients are now living longer than they used to. Cardiac complication has emerged as the leading cause of mortality in DMD patients.³⁷ Embodiments disclosed herein address cardiac benefits (especially the blood-pumping function) in a phenotypic model. In addition, SERCA2a was used instead of SERCA1 because the latter does not express in the heart of wild-type animals. SERCA2a is a better therapeutic target because it can be used to treat both skeletal and cardiac muscle. In addition, a human SERCA2a vector was used that has shown an outstanding safety profile in hundreds of heart failure patients in humans.³¹⁻⁴⁰

Significant improvement was observed in overall body muscle function (forearm grip force and treadmill performance), cardiac electrophysiology (ECG), and heart contractility (hemodynamics). Moreover, SERCA2a therapy effectively prevented myocardial remodeling and fibrosis, although a reduction in skeletal muscle pathology was not observed. Although further studies are needed to clarify this discrepancy, it may likely due to the timing of AAV injection. At 3 months of age, mdx mice show pronounced skeletal muscle disease but no cardiac pathology.^{41,42} SERCA2a therapy may likely act more effectively before the onset, rather than after the onset, of tissue damage. In support of this idea, skeletal muscle histology was significantly improved when SERCA expression was started in utero in transgenic mice or when the AAV SERCA vector was delivered to neonatal mice.^{30,31} Alternatively, delivering SERCA2a to 12-month-old mdx mice did not reduce myocardial fibrosis.³³

Despite the improvement in grip force and treadmill running (FIGS. 4A-4B, FIGS. 5A-5C), an improvement in skeletal muscle histology was not observed (FIGS. 6A-6E, FIGS. 7A-7E). However, muscle function improvement can occur independent of muscle pathology amelioration.⁴⁷

### Methods and Materials

### Experimental Animals

All animal experiments were approved by the Institutional Animal Care and Use Committee and were in accordance with NIH guidelines. C57/BL10 (wild-type control, stock no. 000476) and dystrophin-deficient mdx mice (stock no. 001801) were generated in a barrier facility using breeders purchased from The Jackson Laboratory (Bar Harbor, ME, USA).

The classic presentation of DMD cardiac disease is dilated cardiomyopathy. Cardiac defects have been seen in a variety of DMD mouse models, including mdx, mdx4cv, mdx5cv, D2-mdx, utrophin/dystrophin double-knockout mice, utrophin heterozygous mdx mice, Cmah/dystrophin double-knockout mice, MyoD/dystrophin double-knockout mice, and integrin/dystrophin double-knockout mice. However, dilated cardiomyopathy has only been reported in aged female mdx mice,¹⁷ aged female mdx4cv mice,⁴⁸ and MyoD/dystrophin double-knockout mice.⁴⁹ MyoD/dystrophin double-knockout mice are genetically different from DMD patients. In addition to null mutation in the dystrophin gene, these mice also carry null mutation in the MyoD gene. Furthermore, MyoD/dystrophin double-knockout mice are no longer available. The severity of the cardiac disease is similar between aged female mdx mice and aged female mdx4cv mice. Aged female mdx mice were used.^{16,17} The use of female mice should not reduce translational significance of this study. When the dystrophin gene in both X chromosomes is inactivated in a girl, this girl displays a characteristic DMD phenotype. A therapy that is developed for treating affected boys will be equally effective in treating affected girls and vice versa. The sample size summary for functional assays is shown in TABLE 3.

TABLE 3

| | 8 months | | 18 months | | | |
|---|---|---|---|---|---|---|
| | post-injection | | post-injection | | | |
| | Forelimb | | Forelimb | | | LV |
| | grip | Treadmill | grip | Treadmill | | hemo- |
| | strength | running | strength | running | ECG | dynamics |
| wild-type | 8 | 8 | 11^{a} | n^{a} | 20^{b} | 22^{b} |
| mdx | 8 | 8 | 7 | 7 | 19^{b} | 18^{b} |
| AAV.SERCA2a | 11 | 10^{d} | 9^{e} | 9^{e} | 9^{e} | 6^{f} |

^{a}Three additional age and sex-matched wild type mice became available later and were added to the study.
^{b}This includes historical data from the lab.
^{c}One mdx mouse died before reaching the experimental time point.
^{d}One mouse did not run on the treadmill.
^{e}Two mice died due to accidental animal husbandry issues.
^{f}Two mice died during the surgery and the recording from a third mouse was unusable.

### AAV Production and Delivery

The cis SERCA2a packaging plasmid was modified from a construct published previously.^{33,39} Specifically, a flag tag was fused in-frame to the C terminus of the human SERCA2a cDNA. SERCA2a expression was regulated by the cytomegalovirus promoter, a hybrid intron, and the bovine growth hormone polyadenylation signal. The AAV9 vector was produced, purified, and titrated according to our published protocol.⁵⁰ A total of 6×10¹² vg particles/mouse of the AAV9 SERCA2a vector were injected via the tail vein to conscious 3-month-old mdx mice.

### Morphological Studies

Flag-tagged human SERCA2a was evaluated by immunostaining using a monoclonal antibody against the flag tag (1:500, Sigma-Aldrich, catalog no. F1804, clone M2). Laminin was detected with a polyclonal antibody (1:200, Sigma-Aldrich, catalog no. L9393). General histology was examined by hematoxylin and eosin staining. Fibrosis was examined by Masson trichrome staining.⁵¹ Slides were viewed at the identical exposure setting using a Nikon E800 fluorescence microscope. Photomicrographs were taken with a QImaging Retiga 1300 camera.⁵¹ Fibrotic area in the entire heart section and muscle fiber cross-sectional area (CSA) were quantified using the lasso tool in the Photoshop software on Masson trichrome-stained images.³⁵ Briefly, the micrometer scale was defined with the set measurement scale option in the software. The fibrotic area was marked using the quick selection tool. The sum of all fibrotic areas was then represented as a percentage of the whole-heart CSA. For myofiber CSA measurement, the micrometer scale was defined and the perimeter of each individual fiber was marked using the quick selection tool. The CSA was then calculated by the software.

### SR Calcium Uptake

Calcium uptake in the SR was measured using the Millipore filtration technique.²⁸ Briefly, about 150 mg of the total protein extract was incubated at 37° C. in 1.5 mL of Ca²⁺ uptake medium (40 mmol/L imidazole [pH 7.0], 100 mmol/L KCl, 5 mmol/L MgCl₂, 5 mmol/L NaN₃, 5 mmol/L potassium oxalate, and 0.5 mmol/L EGTA) and various concentrations of CaCl₂) to yield 0.03-3 mmol/L free Ca²⁺ (containing 1 mCi/mmol ⁴⁵Ca²⁺). To obtain the maximal stimulation of SR Ca²⁺ uptake, ruthenium red was added to a final concentration of 1 mmol immediately prior to the addition of the substrates to begin Ca²⁺ uptake. The reaction was initiated by the addition of ATP to a final concentration of 5 mmol and terminated at 1 min by filtration. Each assay was performed in duplicate. The rate of SR Ca²⁺ uptake and the Ca²⁺ concentration required for 50% effective concentration (EC50) were determined by non-linear curve fitting analysis using GraphPad Prism software version 7.0.

### SR Fractionation

SR fraction was prepared at 4° C. unless specified otherwise. Briefly, the tissue (˜25-40 mg) was homogenized in 1 mL of ice-cold buffer A (pH 7.0; 10 mM imidazole, 0.3 M sucrose, 0.5 M dithiothreitol [DTT], 40 mM CaCl₂), and 1× protease inhibitor cocktail; Roche, Indianapolis, IN, USA). The crude lysate was centrifuged at 3,000×g for 20 min. The homogenization and centrifugation steps were repeated once. The supernatant was centrifuged at 10,000×g for 20 min. The resulting supernatant was transferred to a 5-mL Beckman tube and KCl was added to a final concentration of 0.5 mM in buffer A. The lysate was incubated on ice for 20-30 min with occasional agitation. Each sample was then centrifuged at 245,419×g for 40 min. The resulting pellet was resuspended in buffer B (pH 7.5; 20 mM Tris, 0.3 M sucrose, 0.6 M KCl, 0.5 mM DTT, and 40 mM CaCl₂) and centrifuged at 245,419×g for 40 min. The pellet was resuspended in resuspension buffer (pH 7.0; 10 mM imidazole, 0.3 M sucrose, 0.25 mM DTT, and 1× protease inhibitor cocktail). Protein concentration was measured using the DC protein assay kit (Bio-Rad, Hercules, CA, USA).

### Western Blot

Whole heart and muscle lysates were prepared as described previously.¹⁴ Briefly, the tissues were snap-frozen in liquid nitrogen. The frozen tissue samples were ground to fine powder in liquid nitrogen followed by homogenization in a buffer containing 10% sodium dodecyl sulfate, 5 mM ethylenediaminetetraacetic acid, 62.5 mM Tris-HCl at pH 6.8, and the protease inhibitor cocktail (Roche, Indianapolis, IN, USA). The crude lysates were heated at 95° C. for 3 min, chilled on ice for 2 min, and then centrifuged at 16,000×g for 2 min. Supernatant was collected as the whole muscle lysate. Protein concentration was measured using the DC protein assay kit (Bio-Rad, Hercules, CA, USA). The SERCA2a (1:2,500, Badrilla, Leeds, UK, catalog no. A010-235) polyclonal antibody detects both endogenous and human SERCA2a. Expression of human SERCA2a was evaluated using an anti-flag antibody (1:500, Sigma, St. Louis, MO, USA, catalog no. F1804, clone M2). Western blot quantification was performed using the LI-COR Biosciences Image Studio version 5.0.21 software (https://www.licor.com). The intensity of the respective protein band was normalized to the corresponding loading control in the same blot. The relative band intensity was further normalized to the wild-type control. The heart whole-lysate western blot was first conducted using glyceraldehyde 3-phosphate dehydrogenase (1:3,000, Millipore, Billerica, MA, USA, catalog no. MAB374, clone 6C5) as the loading control (FIG. 1G, FIG. 1H). Quantification data from this experiment are shown in FIG. 1D (right panel). Cardiac SERCA2a western blot was repeated using vinculin (1:2,000, Abcam, Cambridge, MA, USA, catalog no. Abl55120) as the loading control. Representative data from this experiment are shown in FIG. 1D (left panel). The skeletal muscle whole-lysate western blot was performed using vinculin as the loading control (FIG. 2A).

### Treadmill Running

A treadmill endurance assay was performed as described previously with modification.⁵² Briefly, mice were subjected to 5-day treadmill acclimation on a 7° uphill treadmill (Columbus Instruments, Columbus, OH, USA). The acclimation protocol began with placing the animal on an unmoving flat treadmill for 2 min, followed by 5 min on a 7° uphill inclined treadmill for each day. All running acclimations were done at 7° on an inclined treadmill only. The first day, the mouse was run at 5 m/min for 15 min followed by 10 m/min for 5 min. On day 2, the mouse was run at 5 m/min for 5 min, 10 m/min for 15 min, and 12 m/min for 5 min in that order. On day 3, the mouse was run at 5 m/min for 5 min, 10 m/min for 15 min, and 12 m/min for 10 min. On days 4 and 5, the mouse was run for 5 m/min for 5 min, 10 m/min for 20 min, 12 m/min for 5 min, and 15 m/min for 5 min. The running distance was measured on day 6. At the day of distance measurement, the mouse was placed on an unmoving treadmill for 2 min and then run at 5 m/min for 5 min. The treadmill speed was then increased by 1 m/min every 5 min. The total running distance was calculated after the mouse became exhausted. Exhaustion is diagnosed when the animal gives up running and ends up in contact with the shocker (at the minimal setting) for typically 1-3 s without attempting to reenter the treadmill. Animals that did not run were excluded from the analysis.

### Serum CK Activity Assay

Fresh serum was collected by tail vein bleeding. The CK activity was determined using a CK liqui-UV test kit from Stanbio Laboratory (Boerne, TX, USA) according to the manufacturer's guidelines.

### Forelimb Grip Strength Measurement

Forelimb grip strength was measured with a computerized grip strength meter (Columbus Instruments, Columbus, OH, USA), as described previously.^{43,53} The grip strength meter has a pulling bar attached to a force transducer and a digital display. The mouse was first acclimated to the apparatus for approximately 5 min. The mouse was then allowed to grab the pulling bar by holding it from the tip of the tail. The mouse was gently pulled away from the grip bar. When the mouse could no longer grasp the bar, the reading was recorded. Protocol was repeated five times with at least 30 s of rest between trials. The highest three values were averaged to obtain the absolute grip strength. Normalized grip strength was obtained by dividing the absolute grip strength by the body weight.

### ECG and Hemodynamic Assay

Cardiac functions were evaluated using published protocols.^{54,55} Specifically, a 12-lead ECG assay was performed using a commercial system from AD Instruments (Colorado Springs, CO, USA).^{51,56} The Q wave amplitude was determined using the lead I tracing. Other ECG parameters were analyzed using the lead II tracing. The QTc interval was determined by correcting the QT interval with the heart rate.⁵⁷ The cardiomyopathy index was calculated by dividing the QT interval by the PQ segment.⁵⁸ Left ventricular hemodynamics was evaluated using a closed chest approach as previously described.^{51,54} The resulting pressure-volume (PV) loops were analyzed with PVAN software (Millar Instruments, Houston, TX, USA). The cardiac relaxation time constant (Tau) was calculated.⁵⁹ The body surface area was calculated.⁶⁰

### Evaluation of Muscle Fiber Type by Immunostaining

Primary myofiber type antibodies were from Developmental Studies Hybridoma Bank at the University of Iowa (DSHB). Specifically, type I myofiber was detected using the primary antibody BA-D5 (1:5) and the secondary antibody Alexa Flour 350 goat anti-mouse IgG2b (1:50, Invitrogen, Cat. No. A21140); type IIa myofiber was detected using the primary antibody SC-71 (1:10) and the secondary antibody Alexa Flour 594 goat anti-mouse IgG1 (1:100, Invitrogen, Cat. No A21125); type IIb myofiber was detected using the primary antibody BF-F3 (1:10) and the secondary antibody FITC goat anti-mouse IgM (1:100, Jackson ImmunoResearch, Cat. No. 115-095-075). Laminin was detected with a polyclonal primary antibody (1:200, Sigma-Aldrich, Cat. No. L9393) and the secondary antibody goat anti-rabbit IgG Alexa Flour Plus 647 (1:100, Invitrogen Cat. No. A32733). For quantification, five random images were taken from each tissue at each fluorescence channel and merged using ImageJ. Counting was done using the multi-point tool in ImageJ. Results were then exported to Excel for percentage calculation.

### Statistical Analysis

Data are presented as mean±standard error of the mean. For all of the physiological assays, data are presented using the scatterplots from individual experimental subjects. One-way ANOVA with Tukey's multiple comparison analysis was performed using GraphPad Prism software version 7.0 for Mac OSX (GraphPad, La Jolla, CA, USA). p<0.05 was considered statistically significant.

### Example 2—In Vitro Human Ventricular Cardiac Tissue Model

Cardiomyocyte Differentiation from DMD Derived iPSC

Human induced pluripotent stem cells (hiPSCs) derived from patients with DMD are direct differentiated into human ventricular cardiomyocytes (hvCMs) and a ventricular subtype yield of over 90% of hiPSC-derived CMs is achieved (Weng, Z., et al. (2014). Stem Cells Dev 23: 1704-1716). In brief, hiPSCs are dissociated and cell clusters are formed in mTeSR1 with 1 ng/ml bone morphogenetic protein 4 (BMP4) overnight in ultra-low-attachment plate and hypoxic condition. From day one to four, cell clusters are treated with 50 μg/ml ascorbic acid (Sigma-Aldrich), 10 ng/ml activin A, 10 ng/ml BMP4, and 10 μM ROCK inhibitor Y-27632 in StemPro-34 medium supplemented by GlutaMAX (Thermo Fisher Scientific) in 5% O₂ hypoxic condition. At day-4 to day-8 post-differentiation, cell clusters in hypoxic condition are treated with 50 μg/ml ascorbic acid and 5 mM IWR-1 in StemPro-34 medium. RPMI 1640 medium with 1×B27 supplement (Thermo Fisher Scientific) containing 50 μg/ml ascorbic acid is used to maintain cell clusters after day-8 in normoxic condition.

Contractile Assessment of Human Ventricular Cardiac Tissue Strips (hvCTS)

3-D multicellular hvCTS myocardial tissues are engineered to assess hvCM contractility and its kinetics by measuring the contractile activities as previously described (Tumbull, I. C., et al. (2014). FASEB J 28: 644-654; and Cashman, T. J., et al., (2016). J Vis Exp 109: e53447). Briefly, cardiospheres from day 15-16 of hiPSC cardiac differentiation are dissociated into single cells and allowed to recover in RPMI 1640 with 1×B27, 50 μg/ml ascorbic acid and 10 μM ROCK inhibitor Y-27632 in the incubator for 3 days before hvCTS construction. Each hvCTS consists of 1.0×10⁶ cardiac cells differentiated from hiPSCs and 1.0×10⁵ human foreskin fibroblasts in a 100 μl ice-cold solution of 2 mg/ml collagen I (Thermo Fisher Scientific), 0.80-0.95 mg/ml Matrigel, 15 mM NaOH, 0.9× Minimum Essential Medium (Sigma-Aldrich), 25 mM 2-[4-(2-hydroxyethyl)piperazin-1-yl]ethanesulfonic acid (HEPES), and 0.1× hvCTS maintenance medium. A volume of 100 μl of the final cell-collagen mixture is then be added to each polydimethylsiloxane (PDMS) bioreactor, consisting of a force-sensing cantilever post at each end of a rectangular well, and returned to the incubator to form the hvCTS attached between the two end-posts. The hvCTS is maintained in DMEM medium supplemented with 10% newborn calf serum (Gibco), with half-media changes every 2 to 3 days, until ready for testing at day 7-8 post-tissue fabrication.

On the day of measurement, the contractile amplitude of hvCTS is measured at 37° C. in hvCTS maintenance medium with HEPES buffer using a custom-designed post-tracking force measurement system that records displacement of the cantilever posts on a temperature-controlled heating plate. During testing, the hvCTS is paced by electrical field stimulation at different frequency. Force generation and contractile kinetics are then be further analyzed by custom-designed data processing and analysis software.

Infection with AAV1.SERCA2a

AAV1.CMV.SERCA2a is a recombinant adeno-associated vector type 1 carrying the CMV promoter driving the cardiac isoform of the sarcoplasmic reticulum calcium ATPase pump (SERCA2a). A solution of AAV1.SERCA2a at a concentration of 1.0×10¹² viral genomes (vg)/ml is used. Each DMD derived human ventricular cardiac tissue strip (hvCTS) is transduced using AAV1.CMV.SERCA2a by adding the following amounts of vector solution: 0.1 μl; 1 μl; 10 μl; or 100 μl. The viral solution stays for 0, 2, 4, 7, and 14 days at which time points all parameters from the human DMD derived hvCTS are measured and compared to Day 0 pre-transduction measurements. In addition, AAV1.GFP (green gluorescent protein) virus on DMD-hvCTS is used as controls for the same time points. Measured parameters include: developed force normalized developed force; rate variability; force variability; force-frequency relationship; and beta adrenergic response. It is expected that DMD-hvCTS with AAV1.GFP to have abnormalities in all the foregoing parameters, and DMD-hvCTS with AAV1.SERCA2a corrects these abnormalities.

hvCTS Contractility Analysis

Side view images of each hvCTS are taken at the measurement day. The height of the PDMS posts and the average tissue height are determined by image analysis of ImageJ (NIH). By using the custom-designed MATLAB data processor executable, the contractile amplitude of the hvCTS measured is converted into force generation. The force data is then summarized, analyzed and visualized by other MATLAB data analysis software.

### Example 3—Clinical Trial to Treat DMD

### Objectives

A phase 2, randomized, double-blind, placebo-controlled trial is performed to evaluate the safety and efficacy of SRD-001 (AAV1/SERCA2a) in subjects with cardiomyopathy secondary to DMD. Primary: to evaluate the safety and efficacy of a one-time intracoronary administration of SRD-001 in subjects with cardiomyopathy secondary to DMD. Secondary: to evaluate the impact of SRD-001 on skeletal muscle function and quality of life.

### Study Design

This phase 2, multi-center, randomized, double-blind, placebo-controlled trial will assess the safety and efficacy of SRD-001 administered as a one-time antegrade epicardial coronary artery infusion for the treatment of anti-AAV1 neutralizing antibody (NAb) negative subjects with cardiomyopathy secondary to DMD. SRD-001 is an adeno-associated virus serotype 1 (AAV1) vector expressing the transgene for sarco(endo)plasmic reticulum Ca2+ ATPase 2a isoform (SERCA2a).

### Methodology

Subjects with a clinical diagnosis of DMD confirmed by genetic testing and evidence of cardiomyopathy will, after giving informed consent, undergo a battery of tests and procedures to determine eligibility and establish baseline measurements of certain parameters within a 30-day screening period prior to randomization. A total of 50 eligible subjects will then be randomized in a 1:1 ratio to either SRD-001 or placebo.

On Day 1, subjects will undergo diagnostic angiography without concomitant left heart catheterization, followed by a one-time antegrade epicardial coronary artery infusion of investigational medicinal product delivered to the 3 major cardiac territories of the left ventricle of the heart (anterior, lateral, inferior/posterior) unless contraindicated. Pre- and post-procedure, subjects will be monitored continuously for a minimum of 4 hours and discharged to home that same day unless peri-procedural complications warrant overnight hospitalization.

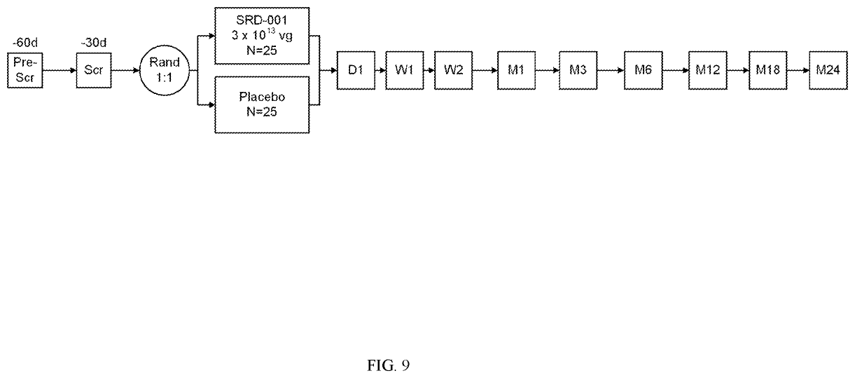

Subjects will be followed during the conduct of the trial at prespecified study visits over a 24-month period. At Week 1, a telephone evaluation will be performed, and if clinically indicated, an in-person evaluation and assessment will be performed at the soonest available time. At Week 2 and Months 1, 3, 6, 12, 18 and 24, subjects will undergo a battery of safety and efficacy assessments including physical examination, laboratory tests, 12-lead electrocardiogram (ECG), collection of adverse events (AEs), and at Months 12 and 24 only, late gadolinium enhancement (LGE) cardiac MRI. Additionally, at Months 6, 12, 18 and 24, subjects will skeletal muscle function as assessed by Performance of the Upper Limb version 2.0 (PUL 2.0), grip strength, key and tip-to-tip pinch strength and elbow flexion strength; if ambulatory, 10-meter Walk/Run Time and North Star Ambulatory Assessment; pulmonary function testing; and quality of life as assessed by DMD Upper Limb Patient-Reported Outcome Measures (DMD UL-PROM) and Pediatric Outcomes Data Collection Instrument (PODCI). The end of the main study for each subject will occur at the Month 24 visit. A schematic of the Phase 2 trial design is depicted FIG. 9.

### Endpoints

Primary efficacy endpoints: change from baseline to Month 12 and Month 24 in left ventricular structure and function as assessed by late gadolinium enhancement (LGE) cardiac MRI including left ventricular ejection fraction, end-diastolic volume, end-systolic volume, stroke volume and circumferential strain; regional wall thickness; left ventricular LGE expressed as a percent of left ventricular mass and in grams; left ventricular viable mass expressed in grams; and number of left ventricular segments with LGE. Alternative primary efficacy endpoint: Composite outcome in change from baseline to Month 12 and Month 24 in LV function (LVESV), PUL 2.0, pulmonary function (pick a parameter), quality of life and terminal events. Secondary efficacy endpoints: change from baseline to Months 6, 12, 18 and 24 in the following: (a) skeletal muscle function as assessed by PUL 2.0, grip strength, key and tip-to-tip pinch strength, elbow flexion strength and, if ambulatory, 10-Meter Walk/Run Time (10MWRT), incidence of loss of ambulation defined as 10MWRT>30 seconds and North Star Ambulatory Assessment (NSAA); (b) pulmonary function as assessed by slow vital capacity (SVC), forced expiratory volume in one second (FEV1), forced vital capacity (FVC), peak expiratory flow (PEF), maximum inspiratory pressure (MIP), maximum expiratory pressure (MEP), peak cough flow (PCF) and inspiratory flow reserve (IFR); and (c) quality of life as assessed by DMD UL-PROM and PODCI.

Primary safety endpoints: incidence of the following from Day 1 through the Month 24 time point: all-cause mortality, serious adverse events, treatment-emergent adverse events related to investigational medicinal product or the administration procedure and cell-mediated immune reaction. Secondary safety endpoints: incidence and severity of all adverse events from Day 1 through the Month 24 time point.

### Diagnosis and Main Criteria for Inclusion

A sample size of 50 is considered an adequate sample size for the primary and secondary objectives of the trial. Number of Subjects (planned): Up to N=50.

Subjects with DMD and evidence of cardiomyopathy will be evaluated for eligibility to participate in the trial as follows: 1. Anti-AAV1 NAb titer<1:2 or equivocal within 60 days of randomization. 2. Male subject at least 10 years of age at time of consent. 3. Willing and able to provide informed consent to participate in the trial if ≥18 years of age, or assent with parental or guardian informed consent if <18 years of age. 4. Diagnosis of DMD based on clinical and phenotypic manifestations consistent with DMD (eg, family history of DMD, elevated creatinine kinase, dystrophin muscle biopsy, calf pseudohypertrophy, Gowers' sign and/or gain impairment before 7 years of age) with confirmatory genetic testing performed at an accredited laboratory. If applicable, loss of independent ambulation by 18th birthday. Standing unassisted or ability to take, at most, several steps independently is not considered ambulation. 5. Cardiomyopathy with left ventricular scar by late gadolinium enhancement in at least 4 segments as assessed by cardiac MRI and left ventricular ejection fraction<40% at the time of screening. 6. Certain PUL 2.0 subjects. 7. If ambulatory, 10-meter walk/run velocity<1 meter/second. 8. Receiving standard-of-care therapy at an experienced, multidisciplinary DMD center with regular cardiac and pulmonary monitoring, systemic glucocorticoid treatment and at-home range-of-motion exercises. 9. Treatment with a systemic glucocorticoid for at least 12 months prior to randomization with a stable dose for at least 6 months prior to randomization with the exception of either weight-based dose adjustment or a decrease in steroid dose of ≤10% for toxicity. 10. Current and up-to-date immunizations, unless contraindicated, including the following: annual influenza; meningococcal and meningococcal B; tetanus, diphtheria & acellular pertussis (Tdap); and pneumococcal polysaccharide vaccinations. 11. Agree to use a condom and spermicide during any sexual relations for 6 months following administration of investigational medicine product to protect their partner from potential viral shedding. 12. Partners capable of procreation must agree to use adequate contraception for 6 months following administration of investigational medicinal product to avoid pregnancy (defined as oral or injectable contraceptives, intrauterine devices, surgical sterilization in addition to/or a combination of a condom and spermicide). 13. Assessed by the investigator as willing and able to comply with the requirements of the investigational plan.

### Main Criteria for Exclusion

Subjects meeting any of the following criteria will be excluded from the study: DMD exclusions such as elbow-flexion contractures>30° in both extremities, body mass index>45, exon 44 skip-amenable mutation(s) in the dystrophin gene, or dystrophin deletion mutation(s) encompassing and limited to exons 307. More criteria for exclusion include: 1. Any IV therapy with positive inotropes, vasodilators or diuretics within 30 days prior to screening or enrollment. 2. Restrictive cardiomyopathy, hypertrophic cardiomyopathy, acute myocarditis, pericardial disease, amyloidosis, infiltrative cardiomyopathy, uncorrected thyroid disease or discrete left ventricular (LV) aneurysm. 3. Cardiac surgery, percutaneous coronary intervention (PCI), valvuloplasty or valve replacement within 30 days prior to screening. 4. Myocardial infarction (e.g., ST elevation MI [STEMI] or large non-STEMI) within 90 days prior to screening. Large non-STEMI shall be defined >3×the upper limit of normal (ULN) for creatine kinase test (CK-MB) or >5×ULN for troponin. 5. Prior heart transplantation, left ventricular reduction surgery (LVRS), cardiomyoplasty, passive restraint device (e.g., CorCap™ Cardiac Support Device), mechanical circulatory support device (MCSD) or cardiac shunt. 6. Likely to receive cardiac resynchronization therapy, cardiomyoplasty, LVRS, conventional revascularization procedure or valvular repair in the 6 months following treatment. 7. Likely need for an immediate heart transplant or MCSD implant due to hemodynamic instability. 8. Known hypersensitivity to radiopaque agents used for angiography; history of or likely need for, high dose corticosteroid pretreatment prior to contrast angiography. 9. Significant left main or ostial right coronary luminal stenosis in the opinion of the investigator, 10. Liver function tests (alanine aminotransferase [ALT], aspartate aminotransferase [AST], alkaline phosphatase)>3×ULN, total bilirubin>2×ULN or known intrinsic liver disease (e.g., cirrhosis, chronic hepatitis B or hepatitis C virus infection). 11. Current or likely need for hemodialysis within 12 months or current glomerular filtration rate (GFR)≤20 mL/minute/1.73 m2 estimated by Modification of Diet in Renal Disease (MDRD) formula for calculating the GFR MDRD calculation. 12. Bleeding diathesis or thrombocytopenia defined as platelet count<75,000 platelets/μL. 13. Anemia defined as hemoglobin<9 g/dL. 14. Inadequate pulmonary function, including the following: a. Percent predicted FVC<35%. b. Inability to perform consistent FVC and PEF measurements within +15%, respectively, during paired testing at screening. c. Risk of near-term respiratory decompensation in the judgment of the investigator, or the need for initiation of non-invasive ventilator support as defined by serum bicarbonate≥29 mmol/L at screening. d. History of non DMD-related chronic respiratory disease requiring ongoing or intermittent treatment including, but not limited to, asthma, bronchitis and tuberculosis. e. Acute respiratory illness within 30 days prior to screening. f. Initiation of non-invasive ventilation within 30 days prior to screening, or the anticipated need to initiate non-invasive ventilation within the 12 months following screening. 15. Planned or anticipated thoracic, spinal or, if ambulatory, lower extremity surgery within 12 months following randomization 16. Initiation of treatment with metformin or insulin within 3 months prior to randomization. 17. Treatment with human growth hormone (HGH) within 3 months prior to randomization, unless on a stable dose for at least 24 months prior to randomization. 18. Diagnosis of, or treatment for, any cancer within the last 5 years except for basal cell carcinoma or carcinomas in situ where surgical excision was considered curative. (Past medical history of cancer is not exclusionary as long as the subject has been disease free for at least 5 years since the time of diagnosis and treatment). 19. Previous participation in a study of gene transfer; however, if the study was unblinded or documentation otherwise exists that the subject was randomized to the placebo control group and did not receive active gene transfer agent, the subject may be considered for this study. 20. Receiving investigational intervention or participating in another clinical study within 30 days or within 5 half-lives of the drug prior to screening. Exception may be made if the individual is enrolled in a non-therapeutic observational study (registry) or the observational portion of a therapeutic study where the sponsoring authority authorizes enrollment. 21. Recent history of psychiatric disease (including drug or alcohol abuse) that is likely to impair subject's ability to comply with protocol-mandated procedures, in the opinion of the investigator. 22. Other concurrent medical condition(s) that, while not explicitly excluded by the protocol, could jeopardize the safety of the subject or objectives of the study.

### Investigational Medicinal Product, Dosage and Mode of Administration

SRD-001 at a dose of 3×10¹³ vg administered into the left and/or right coronary artery via antegrade epicardial coronary artery infusion with commercially available guide or diagnostic cardiac catheters and the B. Braun Perfusor® Space Syringe Pump set at a flow rate of 300 mL/hr over 10 minutes. Nitroglycerin IV infusion should be administered for a minimum of 10 minutes at the highest tolerated dose prior to infusion and concomitantly with the infusion of SRD-001.

### Duration of Treatment

One-time intracoronary infusion.

### Reference Therapy, Dosage and Mode of Administration

Placebo composed of the same excipients as SRD-001 without the AAV1/SERCA2a active ingredient administered into the left and/or right coronary artery via antegrade epicardial coronary artery infusion with commercially available guide or diagnostic cardiac catheters and the B. Braun Perfusor® Space Syringe Pump set at a flow rate of 300 mL/hr over 10 minutes. Nitroglycerin IV infusion should be administered for a minimum of 10 minutes at the highest tolerated dose prior to infusion and concomitantly during infusion.

### Criteria for Evaluation

Efficacy: LGE cardiac MRI, PUL 2.0, grip strength, key and tip-to-tip pinch strength, elbow flexion strength, 10MWRT and NSAA (if ambulatory), pulmonary function, [NT-proBNP] and quality of life as assessed by DMD UL-PROM and PODCI.

Safety: subject disposition, adverse events (AEs), concomitant medications, laboratory tests (complete blood count [CBC] with white blood cell [WBC] differential and platelets, basic metabolic and comprehensive hepatic serum chemistry panels, lactate dehydrogenase [LDH] and uric acid), troponin T, Enzyme-linked ImmunoSpot (ELISpot), urinalysis, physical examination including weight and vital signs and 12-lead electrocardiogram (ECG).

### Statistical Methods

Analysis populations: the intent-to-treat (ITT) population will be all subjects randomized in the trial, summarized and analyzed according to the randomized treatment assignment. The modified ITT (mITT) population will be all subjects actually treated in the trial, summarized and analyzed according to the randomized treatment assignment. The per-protocol (PP) population will be subjects treated in the trial with no protocol violations that significantly impact the completeness, accuracy and/or reliability of the trial data. The safety population will be subjects treated in the trial, summarized and analyzed according to the treatment actually received.

There will be 2 analysis data cutoffs: Month 12: After all randomized subjects have completed the Month 12 visit unless terminated earlier. Month 24: After all randomized subjects have completed the Month 24 visit unless terminated earlier.

Safety and efficacy data will be listed for the ITT population. Safety summaries will be done at Month 12 and Month 24 using the safety population. Efficacy analyses will be done using the mITT population at the Month 12 and 24 analysis data cutoffs. Efficacy analyses will also be done using the ITT and PP populations.

Methods: Summaries will be by treatment group and study visit. Categorical variables will be summarized as frequencies and percentages in each category. Continuous variables will be summarized as numbers of subjects, means, standard deviations, medians and ranges. Clinical event rates will be summarized as events per patient year of follow-up. Treatment effect on clinical event rates will be summarized as hazard ratios as estimated by a semi-parametric joint frailty model to account for competing risk from terminal events.

Both group- and subject-level overall treatment effect will be explored using a composite outcome approach. For the group level analysis, outcomes will be classified into one of the following domains: 1. Cardiac structural parameters: LGE cardiac MRI. 2. Functional parameters: PUL 2.0, grip strength, key and tip-to-tip pinch strength, elbow flexion strength and 10MWRT (in ambulatory subjects only). 3. Pulmonary parameters: pulmonary function parameter. 4. Biomarkers: NT-proBNP (% and absolute change). 5. Quality of life parameters: DMD UL-PROM and PODCI. 6. Clinical Outcome: terminal events.

Treatment effect on changes from baseline will be analyzed using analysis of covariance to control for baseline values or by comparing distributions of change scores using a chi square test of association (or Fisher's exact test if any change category within treatment group contains less than 5 subjects). Treatment effect on clinical outcomes will be analyzed using the joint frailty model. Hypothesis tests will be two sided.

Within treatment groups, a “success” domain will meet the following criteria: for at least one outcome within the domain, SRD-001 superiority is demonstrated at the 0.20 significance level; and for other outcomes within the domain, SRD-001 superiority is demonstrated descriptively.

Group level investigational medicine improvement will be concluded if: there are at least 2 “success” domains; and there is not clinically significant worsening (pre-defined) in the SRD-001 group in any domain.

For the subject-level analysis, each subject will be scored on each outcome as clinically significantly improved (+1), clinically significantly worsened (−1), or unchanged (0) and a total subject level activity score will be calculated as the sum across all outcomes; clinically significant change will be pre-defined. A two-sided t test will be used to test for a treatment effect based on mean scores. Subject-level investigational medicine improvement will be concluded if SRD-001 superiority is demonstrated at the 0.20 significance level.

SRD-001 activity will be considered “significant” if improvement (as defined above) in the SRD-001 group is detected at either the group or subject level; and descriptive improvement in the SRD-001 group is evident at both the group and subject level. Because the efficacy analyses are exploratory, there will be no adjustment to significance levels for multiplicity.

### Schedule of Assessments

A schedule of assessments is depicted in TABLE 3.

TABLE 3

| | Pre- | | | | Day | Week | Week | Month | Month | Month | Month | Month | Month |
|---|---|---|---|---|---|---|---|---|---|---|---|---|---|
| | Screen | Screen | Day 1 | | 2 | 1 | 2 | 1 | 3 | 6 | 12 | 18 | 24 |
| Assessment | ≤60d^{a} | ≤30d^{b} | Pre^{c} | Post^{d} | ±6 h^{e} | ±1 d | ±3 d | ±3 d | ±7 d | ±7 d | ±7 d | ±7 d | ±7 d |
| Informed | ▪ | ▪ | | | | | | | | | | | |
| consent/assent ^{f} | | | | | | | | | | | | | |
| Inclusion/Exclusion | | ▪ | | | | | | | | | | | |
| criteria | | | | | | | | | | | | | |
| Contraindications | | | ▪ | | | | | | | | | | |
| assessment | | | | | | | | | | | | | |
| Medical history | | ▪ | | | | | | | | | | | |
| Demographics | | ▪ | | | | | | | | | | | |
| Concomitant | | ▪ | ▪ | | | | ▪ | ▪ | ▪ | ▪ | ▪ | ▪ | ▪ |
| medications | | | | | | | | | | | | | |
| Physical examination | | ▪ | ▪ | | | | ▪ | ▪ | ▪ | ▪ | ▪ | ▪ | ▪ |
| & vital signs ^{g} | | | | | | | | | | | | | |
| Chest x-ray | | ▪ | | | | | | | | | | | |
| Adverse events ^{h} | ▪ | ▪ | ▪ | ▪ | ▪ | ▪ | ▪ | ▪ | ▪ | ▪ | ▪ | ▪ | ▪ |
| Clinical events ^{h} | | | ▪ | ▪ | ▪ | ▪ | ▪ | ▪ | ▪ | ▪ | ▪ | ▪ | ▪ |
| Anti-AAV1 NAb ⁱ | ▪ | | ▪ | | | | | | | | ▪ | | ▪ |
| ELISpot ^{i, j} | | | ▪ | | | | ▪ | ▪ | ▪ | | | | |
| Hematology ^{i, k} | | ▪ | ▪ | | | | ▪ | ▪ | ▪ | ▪ | ▪ | ▪ | ▪ |
| Serum chemistries ^{i, l} | | ▪ | ▪ | | | | ▪ | ▪ | ▪ | ▪ | ▪ | ▪ | ▪ |
| Troponin T and CK- | | ▪ | ▪ | | | | ▪ | ▪ | ▪ | ▪ | ▪ | ▪ | ▪ |
| MB ⁱ | | | | | | | | | | | | | |
| NT-proBNP ⁱ | | ▪ | ▪ | | | | | | | | ▪ | | ▪ |
| Urinalysis ⁱ | | ▪ | ▪ | | | | ▪ | ▪ | ▪ | ▪ | ▪ | ▪ | ▪ |
| DMD UL-PROM/ | | ▪ | ▪ | | | | | | ▪ | ▪ | ▪ | ▪ | ▪ |
| PODCI | | | | | | | | | | | | | |
| ECG, 12 leads; w/ | | ▪ | ▪ | | | | ▪ | ▪ | ▪ | ▪ | ▪ | ▪ | ▪ |
| interpretation & report | | | | | | | | | | | | | |
| LGE cardiac MRI ^{m} | | ▪ | | | | | | | | | ▪ | | ▪ |
| PU 2.0, grip strength, | | | | | | | | | ▪ | ▪ | ▪ | ▪ | ▪ |
| key & tip-to-tip pinch | | | | | | | | | | | | | |
| strength and elbow | | | | | | | | | | | | | |
| flexion strength | | | | | | | | | | | | | |
| 10-minute walk run test | | ▪ | ▪ | | | | | | ▪ | ▪ | ▪ | ▪ | ▪ |
| (10MWRT)/NSAA | | | | | | | | | | | | | |
| Pulmonary function | | | | | | | | | ▪ | ▪ | ▪ | ▪ | ▪ |
| test | | | | | | | | | | | | | |
| Drug Shipment | | ▪ | | | | | | | | | | | |
| Request/Randomization ⁿ | | | | | | | | | | | | | |
| Coronary | | | ▪ | | | | | | | | | | |
| angiography/drug | | | | | | | | | | | | | |
| administration ^{o} | | | | | | | | | | | | | |
| Telephone evaluation ^{p} | | | | | ▪ | ▪ | ▪ | ▪ | ▪ | | | | |

### Anticipated Outcome

An improvement in one or more of the primary and/or secondary endpoints listed above in the treatment group as compared to the placebo group is observed at one or more of the 6, 12, 18 and 24 month time points.

### Table 3 Abbreviations:

AAV1, adeno-associated virus serotype 1; ALT, alanine aminotransferase; AST, aspartate aminotransferase; BUN, blood urea nitrogen; CBC, complete blood count; CK-MB, creatine kinase test; DMD UL-PROM, DMD Upper Limb Patient-Reported Outcome Measures; d, days; ECG, electrocardiogram; ELISpot, enzyme-linked Immuno-Spot; LGE, late gadolinium enhancement; LDH, lactate dehydrogenase; MRI, magnetic resonance imaging; NAb, neutralizing antibodies; NSAA, North Star Ambulatory Assessment; NT proBNP, N-terminal prohormone brain natriuretic peptide; PODCI, Pediatric Outcomes Data Collection Instrument; PUL 2.0, Performance of the Upper Limb, version 2.0; WBC, white blood cell count. a, prior to screening. b, prior to Randomization. c, prior to administration of investigational medicinal product. d, 2 to 4 hours after administration of investigational medicinal product. e, within 18-30 hours of cardiac catheterization and administration of investigational medicinal product. f, informed consent for the main study is generally obtained not more than 30 days before screening procedures are performed. g, height (screening only), weight, systolic and diastolic blood pressure, pulse and temperature. h, AE/SAE reporting starts when a subject signs the first informed consent for the study and clinical event reporting starts when a subject signs informed consent for the main study. i, performed at a central laboratory. On Day 1, perform any stat lab tests that are required, according to standard of care, for medical clearance for cardiac catheterization and angiography. j, ELISpot may, in addition to the time points indicated, be performed at any time if clinically indicated, k, CBC with WBC differential, hemoglobin, hematocrit and platelet count. 1, basic metabolic panel (Glucose, Sodium, Potassium, Chloride, Bicarbonate, BUN, Creatinine, Calcium) and comprehensive hepatic panel (Albumin, Alkaline Phosphatase, Total Protein, ALT, AST, Direct Bilirubin, Total Bilirubin), LDH and Uric Acid. m, perform cardiac MRI prior to functional tests. n, randomize prior to Day 1 according to recommended shipping lead-time of investigational medicine product. o, includes angiography just prior to infusion, if not performed within preceding 2 months, and co-administration of IV nitroglycerin, as tolerated. p, on Day 2, contact the subject by phone to evaluate possible late complications related to cardiac catheterization (eg, bleeding from the puncture site) and take appropriate action as needed. On Week 1, contact the subject by phone for a general safety assessment; if clinically indicated, an in-person evaluation and assessment should be performed as soon as possible.

### Example 4—In Vivo Study in a Porcine Model

Porcine subjects lacking exon 52 of the DMD gene are a model for DMD, and have a complete loss of dystrophin expression, display a progressive myocardial fibrosis, and have a significantly reduced left ventricular ejection fraction along with ventricular arrhythmias early in lifetime (1-3 months) (Moretti, A. L., et al., (2020), Nat. Med. 26:207-214; Kupatt, C. A., et al., (2021) Gene Ther 28:542-548; and Stirm, M. et al., (2021) Dis Model Mech 14(12)). Subjects were administered AAV1.SERCA2a to evaluate whether AAV1.SERCA2a could treat, reverse or ameliorate some or all of the cardiac abnormalities in the porcine model for DMD.

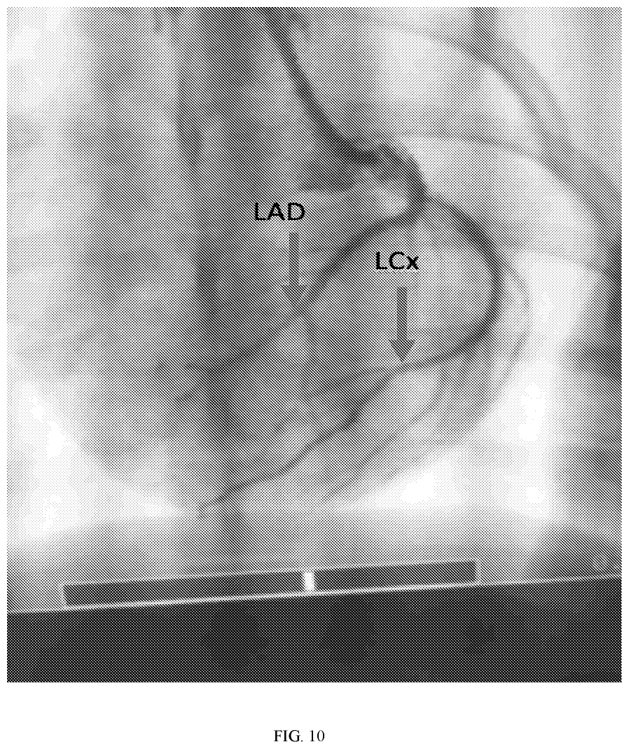

Three subjects were administered AAV1.SERCA2a via the left coronary artery in view of the right porcine right coronary artery begin small and not supplying an appreciable amount of myocardium. In particular, subjects were administered AAV1.SERCA2a at a clinical dose of 3×10¹³ viral genome particles (vg) per subject via slow infusion via intracoronary injection down the left anterior descending coronary artery (LAD) and the left circumflex coronary artery (LCx) (FIG. 10). The AAV1.SERCA2a was diluted in 20 ml of normal saline and was injected via a standard catheter inserted from the left femoral artery. An event recorder was placed behind the left scapula of each subject. During the injections there were no arrhythmias, no blood pressure changes and the subjects tolerated the procedure well.

One month after the injection, the subjects were and were found to have: an improved left ventricular ejection fraction (>25% from baseline); decreased ventricular sizes (decreases of ˜20% from baseline); decreased incidence of ventricular arrhythmias (decreased by 50% from baseline); decreased Myocardial finbrosis throughout the left ventricle (decreased by 50% from baseline); and improved hemodynamics (>15%) as measured by positive dP/dt and negative dP/dt.

These results demonstrated that AAV1.SERCA2a delivered via intracoronary injection improved cardiac function, reversed ventricular dilation, decreased incidence of ventricular arrhythmias and reduced myocardial fibrosis in a large animal model of DMD. These cardiac improvements would lead to improvements in survival in DMD patients.

Each of the following references is incorporated by reference herein in its entirety.

### REFERENCES

1. Burr, A. R., and Molkentin, J. D. (2015). Genetic evidence in the mouse solidifies the calcium hypothesis of myofiber death in muscular dystrophy. Cell Death Differ. 22, 1402-1412.
2. Johnstone, V. P., Viola, H. M., and Hool, L. C. (2017). Dystrophic cardiomyopathy-potential role of calcium in pathogenesis, treatment and novel therapies. Genes (Basel) 8, 108.
3. Allen, D. G., Whitehead, N. P., and Froehner, S. C. (2016). Absence of dystrophin disrupts skeletal muscle signaling: roles of Ca2+, reactive oxygen species, and nitric oxide in the development of muscular dystrophy. Physiol. Rev. 96, 253-305.
4. Vallejo-Illarramendi, A., Toral-Ojeda, I., Aldanondo, G., and López de Munain, A. (2014). Dysregulation of calcium homeostasis in muscular dystrophies. Expert Rev. Mol. Med. 16, e16.
5. Periasamy, M., and Kalyanasundaram, A. (2007). SERCA pump isoforms: their role in calcium transport and disease. Muscle Nerve 35, 430-442.
6. Rossi, A. E., and Dirksen, R. T. (2006). Sarcoplasmic reticulum: the dynamic calcium governor of muscle. Muscle Nerve 33, 715-731.
7. Duan, D. (2016). Systemic delivery of adeno-associated viral vectors. Curr. Opin. Virol. 21, 16-25.
8. Mendell, J. R., Al-Zaidy, S., Shell, R., Arnold, W. D., Rodino-Klapac, L. R., Prior, T. W., Lowes, L., Alfano, L., Berry, K., Church, K., et al. (2017). Single-dose gene-replacement therapy for spinal muscular atrophy. N. Engl. J. Med. 377, 1713-1722.
9. Duan, D. (2018). Micro-dystrophin gene therapy goes systemic in Duchenne muscular dystrophy patients. Hum. Gene Ther. 29, 733-736.
10. Duan, D. (2018). Systemic AAV micro-dystrophin gene therapy for duchenne muscular dystrophy. Mol. Ther. 26, 2337-2356.
11. Chamberlain, J. S., Metzger, J., Reyes, M., Townsend, D., and Faulkner, J. A. (2007). Dystrophin-deficient mdx mice display a reduced life span and are susceptible to spontaneous rhabdomyosarcoma. FASEB J. 21, 2195-2204.
12. Li, D., Long, C., Yue, Y., and Duan, D. (2009). Sub-physiological sarcoglycan expression contributes to compensatory muscle protection in mdx mice. Hum. Mol. Genet. 18, 1209-1220.
13. Lai, Y., Zhao, J., Yue, Y., Wasala, N. B., and Duan, D. (2014). Partial restoration of cardiac function with DPDZ nNOS in aged mdx model of Duchenne cardiomyopathy. Hum. Mol. Genet. 23, 3189-3199.
14. Li, D., Yue, Y., Lai, Y., Hakim, C. H., and Duan, D. (2011). Nitrosative stress elicited by nNOSm delocalization inhibits muscle force in dystrophin-null mice. J. Pathol. 223, 88-98.
15. Thomas, G. D., Sander, M., Lau, K. S., Huang, P. L., Stull, J. T., and Victor, R. G. (1998). Impaired metabolic modulation of alpha-adrenergic vasoconstriction in dystrophindeficient skeletal muscle. Proc. Natl. Acad. Sci. USA 95, 15090-15095.
16. Bostick, B., Yue, Y., Long, C., and Duan, D. (2008). Prevention of dystrophin-deficient cardiomyopathy in twenty-one-month-old carrier mice by mosaic dystrophin expression or complementary dystrophin/utrophin expression. Circ. Res. 102, 121-130.
17. Bostick, B., Yue, Y., and Duan, D. (2010). Gender influences cardiac function in the mdx model of Duchenne cardiomyopathy. Muscle Nerve 42, 600-603.
18. Chamberlain, J. R., and Chamberlain, J. S. (2017). Progress toward gene therapy for Duchenne muscular dystrophy. Mol. Ther. 25, 1125-1131.
19. Duan, D. (2018). CRISPR alleviates muscular dystrophy in dogs. Nat. Biomed. Eng. 2, 795-796.
20. Min, Y. L., Bassel-Duby, R., and Olson, E. N. (2019). CRISPR correction of Duchenne muscular dystrophy. Annu. Rev. Med. 70, 239-255.
21. Mendell, J. R., Campbell, K., Rodino-Klapac, L., Sahenk, Z., Shilling, C., Lewis, S., Bowles, D., Gray, S., Li, C., Galloway, G., et al. (2010). Dystrophin immunity in Duchenne's muscular dystrophy. N. Engl. J. Med. 363, 1429-1437.
22. Bellinger, A. M., Reiken, S., Carlson, C., Mongillo, M., Liu, X., Rothman, L., Matecki, S., Lacampagne, A., and Marks, A. R. (2009). Hypernitrosylated ryanodine receptor calcium release channels are leaky in dystrophic muscle. Nat. Med. 15, 325-330.
23. Michelucci, A., Garcia-Castañeda, M., Boncompagni, S., and Dirksen, R. T. (2018). Role of STIM1/ORAI1-mediated store-operated Ca2+ entry in skeletal muscle physiology and disease. Cell Calcium 76, 101-115.
24. Anand, R., and Emery, A. E. (1982). Verapamil and calcium-stimulated enzyme efflux from skeletal muscle. Clin. Chem. 28, 1482-1484.
25. Phillips, M. F., and Quinlivan, R. (2008). Calcium antagonists for Duchenne muscular dystrophy. Cochrane Database Syst. Rev. (4), CD004571.
26. Capogrosso, R. F., Mantuano, P., Uaesoontrachoon, K., Cozzoli, A., Giustino, A., Dow, T., Srinivassane, S., Filipovic, M., Bell, C., Vandermeulen, J., et al. (2018). Ryanodine channel complex stabilizer compound 548168/ARM210 as a diseasemodifier in dystrophin-deficient mdx mice: proof-of-concept study and independent validation of efficacy. FASEB J. 32, 1025-1043.
27. Hoshijima, M., Ikeda, Y., Iwanaga, Y., Minamisawa, S., Date, M. O., Gu, Y., Iwatate, M., Li, M., Wang, L., Wilson, J. M., et al. (2002). Chronic suppression of heart-failure progression by a pseudophosphorylated mutant of phospholamban via in vivo cardiac rAAV gene delivery. Nat. Med. 8, 864-871.
28. Voit, A., Patel, V., Pachon, R., Shah, V., Bakhutma, M., Kohlbrenner, E., McArdle, J. J., Dell'Italia, L. J., Mendell, J. R., Xie, L. H., et al. (2017). Reducing sarcolipin expression mitigates Duchenne muscular dystrophy and associated cardiomyopathy in mice. Nat. Commun. 8, 1068.
29. Tanihata, J., Nagata, T., Ito, N., Saito, T., Nakamura, A., Minamisawa, S., Aoki, Y., Ruegg, U. T., and Takeda, S. (2018). Truncated dystrophin ameliorates the dystrophic phenotype of mdx mice by reducing sarcolipin-mediated SERCA inhibition. Biochem. Biophys. Res. Commun. 505, 51-59.
30. Goonasekera, S. A., Lam, C. K., Millay, D. P., Sargent, M. A., Hajjar, R. J., Kranias, E. G., and Molkentin, J. D. (2011). Mitigation of muscular dystrophy in mice by SERCA overexpression in skeletal muscle. J. Clin. Invest. 121, 1044-1052.
31. Morine, K. J., Sleeper, M. M., Barton, E. R., and Sweeney, H. L. (2010). Overexpression of SERCA1a in the mdx diaphragm reduces susceptibility to contraction-induced damage. Hum. Gene Ther. 21, 1735-1739.
32. Mizala, D. A. G., Pratt, S. J. P., Chen, D., Molkentin, J. D., Lovering, R. M., and Chin, E. R. (2015). SERCA1 overexpression minimizes skeletal muscle damage in dystrophic mouse models. Am. J. Physiol. Cell Physiol. 308, C699-C709.
33. Shin, J.-H., Bostick, B., Yue, Y., Hajjar, R., and Duan, D. (2011). SERCA2a gene transfer improves electrocardiographic performance in aged mdx mice. J. Transl. Med. 9, 132.
34. Dutta, S., and Sengupta, P. (2016). Men and mice: relating their ages. Life Sci. 152, 244-248.
35. Wasala, N. B., Lai, Y., Shin, J.-H., Zhao, J., Yue, Y., and Duan, D. (2016). Genomic removal of a therapeutic mini-dystrophin gene from adult mice elicits a Duchenne muscular dystrophy-like phenotype. Hum. Mol. Genet. 25, 2633-2644.
36. Lai, Y., and Duan, D. (2012). Progress in gene therapy of dystrophic heart disease. Gene Ther. 19, 678-685.
37. McNally, E. M., Kaltman, J. R., Benson, D. W., Canter, C. E., Cripe, L. H., Duan, D., Finder, J. D., Groh, W. J., Hoffman, E. P., Judge, D. P., et al.; Working Group of the National Heart, Lung, and Blood Institute; Parent Project Muscular Dystrophy (2015). Contemporary cardiac issues in Duchenne muscular dystrophy. Circulation 131, 1590-1598.
38. Greenberg, B., Butler, J., Felker, G. M., Ponikowski, P., Voors, A. A., Desai, A. S., Barnard, D., Bouchard, A., Jaski, B., Lyon, A. R., et al. (2016). Calcium upregulation by percutaneous administration of gene therapy in patients with cardiac disease (CUPID 2): a randomised, multinational, double-blind, placebo-controlled, phase 2b trial. Lancet 387, 1178-1186.
39. Zsebo, K., Yaroshinsky, A., Rudy, J. J., Wagner, K., Greenberg, B., Jessup, M., and Hajjar, R. J. (2014). Long-term effects of AAV1/SERCA2a gene transfer in patients with severe heart failure: analysis of recurrent cardiovascular events and mortality. Circ. Res. 114, 101-108.
40. Jaski, B. E., Jessup, M. L., Mancini, D. M., Cappola, T. P., Pauly, D. F., Greenberg, B., Borow, K., Dittrich, H., Zsebo, K. M., and Hajjar, R. J.; Calcium Up-Regulation by Percutaneous Administration of Gene Therapy in Cardiac Disease (CUPID) Trial Investigators (2009). Calcium upregulation by percutaneous administration of gene therapy in cardiac disease (CUPID trial), a first-in-human phase 1/2 clinical trial. J. Card. Fail. 15, 171-181.
41. Bostick, B., Yue, Y., Lai, Y., Long, C., Li, D., and Duan, D. (2008). Adeno-associated virus serotype-9 microdystrophin gene therapy ameliorates electrocardiographic abnormalities in mdx mice. Hum. Gene Ther. 19, 851-856.
42. De la Porte, S., Morin, S., and Koenig, J. (1999). Characteristics of skeletal muscle in mdx mutant mice. Int. Rev. Cytol. 191, 99-148.
43. Li, D., Yue, Y., and Duan, D. (2008). Preservation of muscle force in Mdx3cv mice correlates with low-level expression of a near full-length dystrophin protein. Am. J. Pathol. 172, 1332-1341.
44. Judge, L. M., Arnett, A. L., Banks, G. B., and Chamberlain, J. S. (2011). Expression of the dystrophin isoform Dp116 preserves functional muscle mass and extends lifespan without preventing dystrophy in severely dystrophic mice. Hum. Mol. Genet. 20, 4978-4990.
45. Baltgalvis, K. A., Jaeger, M. A., Fitzsimons, D. P., Thayer, S. A., Lowe, D. A., and Ervasti, J. M. (2011). Transgenic overexpression of g-cytoplasmic actin protects against eccentric contraction-induced force loss in mdx mice. Skelet. Muscle 1, 32.
46. Bogdanovich, S., McNally, E. M., and Khurana, T. S. (2008). Myostatin blockade improves function but not histopathology in a murine model of limb-girdle muscular dystrophy 2C. Muscle Nerve 37, 308-316.
47. Duan, D. (2019). Considerations on preclinical neuromuscular disease gene therapy design. In Muscle Gene Therapy, Second Edition, D. Duan and J. R. Mendell, eds. (Springer Nature Switzerland), pp. 291-326.
48. Wasala, N. B., Yue, Y., Vance, J., and Duan, D. (2017). Uniform low-level dystrophin expression in the heart partially preserved cardiac function in an aged mouse model of Duchenne cardiomyopathy. J. Mol. Cell. Cardiol. 102, 45-52.
49. Megeney, L. A., Kablar, B., Perry, R. L., Ying, C., May, L., and Rudnicki, M. A. (1999). Severe cardiomyopathy in mice lacking dystrophin and MyoD. Proc. Natl. Acad. Sci. USA 96, 220-225.
50. Shin, J.-H., Yue, Y., and Duan, D. (2012). Recombinant adeno-associated viral vector production and purification. Methods Mol. Biol. 798, 267-284.
51. Wasala, N. B., Bostick, B., Yue, Y., and Duan, D. (2013). Exclusive skeletal muscle correction does not modulate dystrophic heart disease in the aged mdx model of Duchenne cardiomyopathy. Hum. Mol. Genet. 22, 2634-2641.
52. Bostick, B., Yue, Y., Long, C., Marschalk, N., Fine, D. M., Chen, J., and Duan, D. (2009). Cardiac expression of amini-dystrophin that normalizes skeletal muscle force only partially restores heart function in aged dmx mice. Mol. Ther. 17, 253-261.
53. Hakim, C. H., Li, D., and Duan, D. (2011). Monitoring murine skeletal muscle function for muscle gene therapy. Methods Mol. Biol. 709, 75-89.
54. Bostick, B., Yue, Y., and Duan, D. (2011). Phenotyping cardiac gene therapy in mice. Methods Mol. Biol. 709, 91-104.
55. Duan, D., Rafael-Fortney, J. A., Blain, A., Kass, D. A., McNally, E. M., Metzger, J. M., Spumey, C. F., and Kinnett, K. (2016). Standard operating procedures (SOPs) for evaluating the heart in preclinical studies of Duchenne muscular dystrophy. J. Cardiovasc. Transl. Res. 9, 85-86.
56. Bostick, B., Shin, J.-H., Yue, Y., and Duan, D. (2011). AAV-microdystrophin therapy improves cardiac performance in aged female mdx mice. Mol. Ther. 19, 1826-1832.
57. Mitchell, G. F., Jeron, A., and Koren, G. (1998). Measurement of heart rate and Q-T interval in the conscious mouse. Am. J. Physiol. 274, H747-H751.
58. Nigro, G., Comi, L. I., Politano, L., and Nigro, G. (2004). Cardiomyopathies associated with muscular dystrophies. In Myology. Basic and Clinical, Third Edition, Vol. 2, A. Engel and C. Franzini-Armstrong, eds. (McGraw-Hill), pp. 1239-1256.
59. Weiss, J. L., Frederiksen, J. W., and Weisfeldt, M. L. (1976). Hemodynamic determinants of the time-course of fall in canine left ventricular pressure. J. Clin. Invest. 58, 751-760.
60. Cheung, M. C., Spalding, P. B., Gutierrez, J. C., Balkan, W., Namias, N., Koniaris, L. G., and Zimmers, T. A. (2009). Body surface area prediction in normal, hypermuscular, and obese mice. J. Surg. Res. 153, 326-331.

The term “comprising” as used herein is synonymous with “including,” “containing,” or “characterized by,” and is inclusive or open-ended and does not exclude additional, unrecited elements or method steps.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1. A method of treating, inhibiting or ameliorating a skeletal muscular dystrophy in a subject, comprising:
administering a polynucleotide comprising a nucleic acid encoding a sarcoplasmic/endoplasmic reticulum calcium ATPase (SERCA) polypeptide to the subject.

2.-111. (canceled)
